(19) United States
(12) Patent Application Publication
(10) Pub. No.: US 2005/0101530 A1
(43) Pub. Date: May 12, 2005

(54) Cellular virus receptors and methods of use

(75) Inventors: Paolo Renzi, Westmount (CA); Khalid Zemzoumi, Montreal (CA)

Correspondence Address: DAVID S. RESNICK, 100 SUMMER STREET, NIXON PEABODY LLP, BOSTON, MA, 02110-2131, US

(73) Assignee: Topigen Pharmaceutique, Inc., Montreal, QC (CA)

(21) Appl. No.: 10/486,471

(22) PCT Filed: Aug. 12, 2002
(86) PCT No.: PCT/CA02/01248

Related U.S. Application Data
(60) Provisional application No. 60/311,088, filed on Aug. 10, 2001.

(51) Int. Cl. A61K 38/17; C12Q 1/70

(52) U.S. Cl. 514/12; 435/5

(57) ABSTRACT

Methods, reagents and compositions for the treatment, prevention and diagnotic of virus infections in vertebrates and more paticularly in human and animals are described. The invention provides evidence that the CCR1, CCR2, CCR3, CCR4, CCR5 and CCR8 receptors are involved in human respiratory syncytial virus (RSV) infections. Therefore, the present invention describes methods for modulation of cellular viral infection by modulating a binding interaction between a CCR1, CCR2, CCR3, CCR4, CCR5 and/or CCR8 receptor and a surface protein of the virus. The invention also profits of such a binding interaction for 10 providing methods for reducing viral infection of a cell; methods of attenuating the ability of a pneumovirus to bind a mammalian cell; methods for reducing the initiation or spread of a respiratory tract disease due to human RSV; methods for detecting the presence of a pneumovirus in a biological sample; gene therapy methods, and methods for identifying novel antiviral and anti-inflammatory 15 compounds.

### RELATED APPLICATION

This application claims priority of U.S. Provisional Application 60/311,088 filed Aug. 10, 2001, the disclosure of which is incorporated by reference herein in its entirety.

### BACKGROUND OF THE INVENTION

1. (a) Field of the Invention The invention relates to methods, reagents and compositions for treating or preventing respiratory virus infections in vertebrates, and more particularly, respiratory syncytial virus (RSV) infections in human and animals. The invention also relates to methods for modulating viral infection of cells by modulating a binding interaction between a CCR1, CCR2, CCR3, CCR4, CCR5 and/or CCR8 receptor and a surface protein of the virus. The invention also profits of such a binding interaction in methods for reducing viral infection of a cell; in methods of attenuating the ability of a pneumovirus to bind a mammalian cell; methods for reducing the initiation or spread of a respiratory tract disease due to human RSV; in methods for detecting the presence of a pneumovirus in a biological sample; in gene therapy methods, and in methods for identifying novel antiviral compounds.

2. (a) Brief Description of the Prior Art

Human Respiratory Syncytial Virus (HRSV) is a non-segmented, negative-strand virus in the pneumovirus subfamily of Paramyxoviridae. The virus has been described in detail by Collins, P. et al. in the textbook by Fields, B. N. et al., (1996) Fields Virology, pp 1313-1351 (Raven Press, N.Y). The virus is ubiquitous in the human population and approximately 100% of infants are infected by the age of 3. HRSV infection is the leading cause of serious lower respiratory tract disease in infants and children: HRSV is responsible for at least 50% of bronchiolitis hospitalizations, 25% of pneumonia hospitalizations, and 2% mortality rate among hospitalized infants annually. Approximately 50% of bronchiolitis patients develop asthma. In adults 60 years or older, HRSV causes symptoms similar to the common cold or flu, however it may also cause pneumonia, bronchitis, and death. Although HRSV is thought to account for over 1 million deaths per year worldwide, there is no effective and safe HRSV vaccine currently available. The World Health Organization and National Institutes of Allergy and Infectious Diseases vaccine advisory committees have ranked HRSV second to HIV for vaccine development.

Viruses infect cells by interacting with one or more specific cellular receptor proteins that function as coreceptors for the virus (also called virus receptors), as a portal of entry for the virus to gain entry into the target host cells. It is known that HRSV specifically infects alveolar cell types including macrophages and epithelial cells of the respiratory system. The G- and the F-glycoproteins are the two major proteins of the HRSV envelope that are known to be involved in HRSV infection, the attachment (G) glycoprotein being responsible in part for the entry of the virus into the host cells. Recently, it has been demonstrated that HRSV binds the receptor CX3CR1 (the specific receptor for the chemokine fraktalkine) and that this binding facilitates RSV infection of cells (Tripp et al. (2001) Nature Immunology, 8:732-738). The present invention discloses additional cell receptors (CCR1, CCR2, CCR3, CCR4, CCR5, and CCR8) which recognize and bind the RSV-G protein, these additional receptors being involved in HRSV entry into the cell.

Several vertebrate cell receptors play a critical role in the pathogenesis of certain viral, bacterial and parasite infections, and have been termed coreceptors (or co-receptors). For example, chemokine receptors CCR2b, CCR3, CCR5, and CXCR4, and others, are involved in HIV infection and their corresponding ligands (chemokines) can also inhibit virus entry and infection (Pelchen-Mathews et al., (1999), Immunological Reviews 168: 3349; Springer et al. (2001) J of Virol 75: 3779-90; and PCT patent application WO 99/06561). Other types of cell receptors also function as coreceptors/viral receptors. For example, the ICAM-1 receptor involved in cellular adhesion is also a coreceptor for human rhinovirus (U.S. Pat. No. 5,589,453). Although the CCR1, CCR2, CCR3, CCR4, CCR5 and CCR8 receptors are well known and have been described in detail previously, they have never been identified up to date as the host cell receptor for RSV, nor for any other virus of the same order (Mononegavirales).

In view of the above, there is a need for identifying the viral cell receptor(s) of viruses of the Mononegavirales order, and more particularly for Pneumoviruses such as the human RSV.

There is also a long felt need for safe and effective RSV vaccines and compositions for modulating viral cellular infection.

There is also a need for methods, reagents and compositions for treating or preventing pneumovirus infections in vertebrates and more particularly respiratory syncytial virus infections in mammals.

There is also a need for methods for identifying novel antiviral compounds for viruses of the Mononegavirales order, and more particularly Pneumoviruses such as the human RSV.

The present invention fulfils these needs and also other needs which will be apparent to those skilled in the art upon reading the following description.

### SUMMARY OF THE INVENTION

The present invention provides methods, compositions and kits for treating, preventing and/or detecting respiratory infections from viruses, and more particularly Pneumoviruses such as the human RSV.

The invention further provides methods and tools for identifying/screening in vitro, in vivo and ex vivo, novel antiviral compounds, drugs and vaccine,

The present invention also provides gene therapy and transfection methods wherein viruses are used as a delivery vehicle for transferring an exogenous gene in CCR1, CCR2, CCR3, CCR4, CCR5 and/or CCR8-positive cells.

According to a first aspect, the invention relates to a method for modulating viral infection of a cell, comprising modulating a binding interaction between a cell chemokine-receptor and a surface protein of the virus, the cell chemokine-receptor comprising an amino acid sequence having at least 38% identity with SEQ ID NO:6. More preferably, the cell chemokine-receptor consists of the CCR1, CCR2, CCR3, CCR4, CCR5, and/or CCR8.

According to another aspect, the invention relates to a method for modulating viral infection of a cell, comprising modulating a binding interaction between a cell chemokine-receptor and a surface protein of said virus. According to the invention, the cell chemokine-receptor is the CCR1, CCR2, CCR3, CCR4, CCR5, and/or the CCR8 and the virus is not HIV.

According to a further aspect, the invention relates to a method for increasing viral infection of a cell comprising permitting or increasing a binding interaction between at least one chemokine-receptor of the cell and a surface protein of the virus. According to a related aspect, the invention relates to a method for reducing viral infection of a cell, comprising interfering with a binding interaction between at least one chemokine-receptor of the cell and a surface protein of the virus. In both cases, the chemokine-receptor is selected from the group consisting of CCR1, CCR2, CCR3, CCR4, CCR5, and CCR8.

Preferably, the virus consists of a virus of the order Mononegavirales. More preferably, the virus consists of a virus of the order the family Paramyxoviridae. Even more preferably, the virus consists of a virus of the Pneumovirinae subfamily, or a virus of the Pneumoviruses species. In most preferred embodiments, the virus consists of the Human Respiratory Syncytial Virus (HRSV) the viral protein consists of the HRSV G-glycoprotein.

In more specific aspects, the invention concerns:
a method for reducing pneumoviral infection of a CCR1-positive cell, comprising interfering with binding of a pneumovirus to CCR1 receptor(s) of the cell;
a method for reducing pneumoviral infection of a CCR2-positive cell, comprising interfering with binding of a pneumovirus to CCR2 receptor(s) of the cell;
a method for reducing pneumoviral infection of a CCR3-positive cell, comprising interfering with binding of a pneumovirus to CCR3 receptor(s) of the cell;
a method for reducing pneumoviral infection of a CCR4-positive cell, comprising interfering with binding of a pneumovirus to CCR4 receptor(s) of the cell;
a method for reducing pneumoviral infection of a CCR5-positive cell, comprising interfering with binding of a pneumovirus to CCR5 receptor(s) of the cell; and
a method for reducing pneumoviral infection of a CCR8-positive cell, comprising interfering with binding of a pneumovirus to CCR8 receptor(s) of the cell.

In another specific aspect, the invention concerns a method for modulating a pneumovirus infection of a cell, comprising modulating a binding interaction between a cell CCR1, CCR2, CCR3, CCR4, CCR5, and/or CCR8 receptor and a surface protein of the pneumovirus.

Therefore, the present invention describes methods for modulation of viral infection of cell by modulating a binding interaction between a CCR1, CCR2, CCR3, CCR4, CCR5 and/or CCR8 receptor and a surface protein of the virus.

The invention also profits of the binding interaction between the CCR1, CCR2, CCR3, CCR4, CCR5 and/or CCR8 receptor and surface protein(s) of viruses for providing methods for reducing viral infection of a cell; methods of attenuating the ability of a pneumovirus to bind a mammalian cell; methods for reducing the initiation or spread of a respiratory tract disease due to human RSV; methods for detecting the presence of a pneumovirus in a biological sample; gene therapy methods, and methods for identifying novel antiviral compounds.

Identification of the CCR1, CCR2, CCR3, CCR4, CCR5 and CCR8 as being coreceptors for HRSV has numerous advantages since it is known that it is the cell receptor expression which determines the tropism of a virus and hence plays a key role in determining virus pathogenicity. Furthermore, the characterization of virus cell receptor(s) facilitates the design and identification of vaccines and antiviral therapeutic agents that may prevent the infection and even treat the infection.

Other objects and advantages of the present invention will be apparent upon reading the following non-restrictive description of several preferred embodiments made with reference to the accompanying drawings.

### BRIEF DESCRIPTION OF THE DRAWINGS

FIG. 1 illustrates that CCR3 is a receptor for the RSV-G protein in Hep-2 cells as demonstrated by coprecipitation of RSV-G protein and CCR3 receptor protein using immunoprecipitation followed by Western blotting.

FIG. 2 illustrates that addition of eotaxin (a ligand for CCR3) to culture medium inhibited RSV infection most effectively early in infection, during virus adsorption to Hep-2 cells.

FIGS. 3A, 3B, 3C, and 3D illustrate that CCR3 transfected GHOST cells support RSV infection and produce infectious virus as demonstrated 48 hours after infection by immunostainning. FIG. 3A shows that GHOST cells that are transfected with the CCR3 receptor are infected by RSV. FIG. 3B demonstrates that addition of eotaxin (1 μg/ml) to GHOST cells that are transfected with the CCR3 receptor inhibits cellular infection by RSV. FIGS. 3C and 3D show that GHOST cells that are not transfected with any chemokine receptor do not support viral infection. These data show that initial contact between RSV and these cells occurs specifically through binding to the CCR3 receptor.

FIGS. 4A and 4B (infected and control non infected, respectively) illustrate that RSV infection occurs also in Hep-2 cells and infectious virus is produced 48 hours after infection as demonstrated by immunostaining. FIGS. 4C and 4D illustrate the neutralizing effect of antibodies directed against the eotaxin receptor (CCR3) on RSV infection. Infection of GHOST cells transfected with the CCR3 receptor by RSV (FIG. 4C) was blocked by neutralizing antibodies specific for CCR3 (FIG. 4D).

FIGS. 5A (non infected) and 5B (infected) illustrate that A549 epithelial cells support RSV infection and produce infectious virus as demonstrated 48 hours after infection by immunostaining. FIGS. 5C and 5D illustrate the neutralizing effect of antibodies directed against the eotaxin receptor (CCR3) on RSV infection. Infection of A549 cells by RSV was blocked by neutralizing antibodies against the CCR3 receptor (FIG. 5C) but was not inhibited by neutralizing antibodies against the CCR5 receptor (FIG. 5D).

FIG. 6A is a sequence alignment of the RSV-G chemokine domain with the human chemokine fraktalkine domain. FIG. 6B is a sequence alignment of the chemokine receptor N-terminal regions. FIG. 6C is a sequence alignment of the extracellular domain 1 (EC1) of the chemokine receptors. FIG. 6D is a sequence alignment of the chemokine receptors extracelular 2 domain (EC2). FIG. 6E is a sequence alignment of chemokine receptors extracellular domain 3 (EC3).

FIG. 7 is a picture of a Western-blot illustrating that GHOST cell lines expressing the chemokine receptor CCR4 or CCR5 support RSV infection. MW: molecular weight; CCR4c or CCR5c: non infected cells; CCR4i or CCR5i: infected cells. The arrow indicates the position of the 45 kDa glycosylated form of RSV-G protein recognized by the antibodies used for the experiment.

FIG. 8 is a picture of a Western-blot illustrating that GHOST cell lines expressing the chemokine receptor CCR1, CCR2 or CCR3 support RSV infection. Lane1: molecular weight; Lane2: CCR3 (infected cells); Lane3: CCR3 (control); Lane4: CCR1 (infected cells); Lane5: CCR1 (control); Lane6: CCR2b (infected cells); Lane7: CCR2b (control). The arrow indicates the position of the 45 kDa glycosylated form of RSV-G protein recognized by the antibodies used for the experiment.

### DETAILED DESCRIPTION OF THE INVENTION

The present invention is based on the discovery that the CCR3 receptor, and also the CCR1, CCR2, CCR4, CCR5 and CCR8 are cellular coreceptors for viruses from the Paramyxoviridae family, and more particularly coreceptors for the human respiratory syncytial virus (HRSV), a member of Pneumoviridae subfamily.

The discovery that the CCR1, CCR2, CCR3 CCR4, CCR5 and CCR8 are cellular receptors for the human respiratory syncytial virus (RSV) and that the RSV-G protein does bind to at least one of these chemokine receptors, has numerous applications, in vitro as well as ex vivo and in vivo.

A) Definitions

Throughout the text, the word “kilobase” is generally abbreviated as “kb”, the words “deoxyribonucleic acid” as “DNA”, the words “ribonucleic acid” as “RNA”, the words “complementary DNA” as “cDNA”, the words “polymerase chain reaction” as “PCR”, and the words “reverse transcription” as “RT”. Nucleotide sequences are written in the 5′ to 3′ orientation unless stated otherwise.

In order to provide an even clearer and more consistent understanding of the specification and the claims, including the scope given herein to such terms, the following definitions are provided:

Antisense: Refers to nucleic acid molecules capable of regulating the expression of a corresponding gene in humans and animals. An antisense molecule as used herein may also encompass a gene construct comprising a structural genomic gene, a cDNA gene or part thereof in reverse orientation relative to its or another promoter. Typically antisense nucleic acid sequences are not templates for protein synthesis but yet interact with complementary sequences in other molecules (such as a gene or RNA), thereby causing the function of those molecules to be affected.

Binding interaction/Binding affinity: Refers to quality, state or process of the attraction or adherence of two molecules to one another. Typically, binding occurs because the shape and chemical natures of parts of the molecules surfaces are complementary and/or have a relatively high attraction or affinity for each other. As used herein, it generally refers to the binding of a surface protein of a virus to a receptor of a host cell susceptible to infection by the virus.

CCR-ligand: Refers to any molecule having a binding affinity for any of the CCR1, CCR2, CCR3, CCR4, CCR5, and/or CCR8 receptor. Preferably, the binding affinity of the CCR-ligand is such that it is capable of blocking a binding interaction between a viral surface protein and a corresponding CCR receptor. A non limitative list of specific examples of CCR-ligands is given hereinafter.

Expression: refers to the process by which gene encoded information is converted into the structures present and operating in the cell. In the case of cDNAs, cDNA fragments and genomic DNA fragments, the transcribed nucleic acid is subsequently translated into a peptide or a protein in order to carry out its function if any.

Fragment: Refers to a section of a molecule, such as a protein, a polypeptide or a nucleic acid, and is meant to refer to any portion of the amino acid or nucleotide sequence.

Homolog: refers to a nucleic acid molecule or polypeptide that shares similarities in DNA or protein sequences.

Modulation: Refers to the process by which a given variable is regulated to a certain proportion.

Specifically binds: means an antibody that recognizes and binds a protein but that does not substantially recognize and bind other molecules in a sample, e.g., a biological sample, that naturally includes protein.

Virus-ligand: Refers to any molecule having a binding affinity for viral surface proteins capable of binding any of the CCR1, CCR2, CCR3, CCR4, CCR5, and/or CCR8 receptor. Preferably, the binding affinity of the virus-ligand is such that it is capable of blocking a binding interaction between a given viral surface protein and a corresponding CCR receptor. A non limitative list of specific examples of virus-ligands is given hereinafter.

B) Chemokine Receptors CCR1, CCR2, CCR3, CCR4, CCR5 and CCR8 are Cellular Coreceptors for HRSV

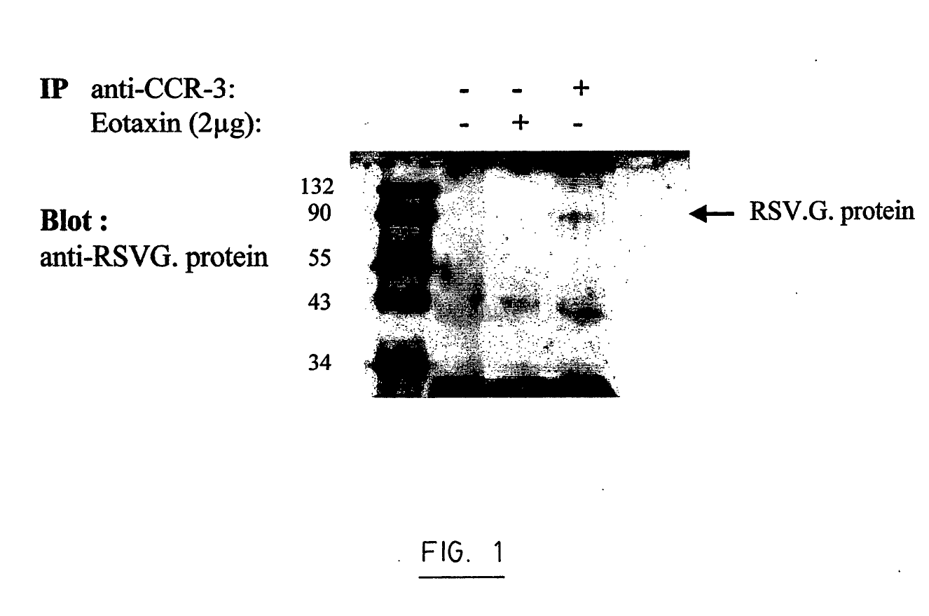

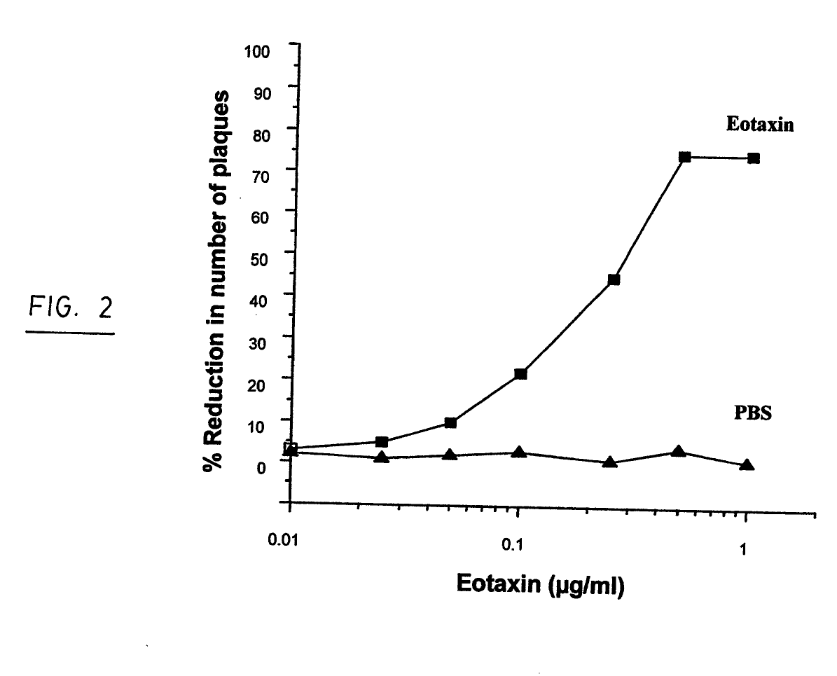

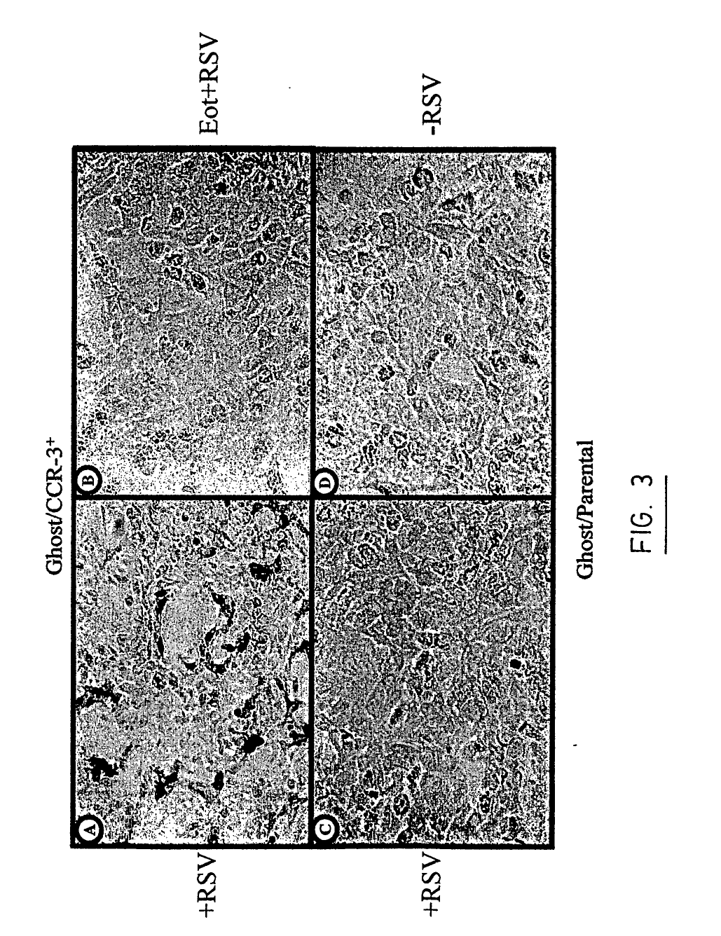

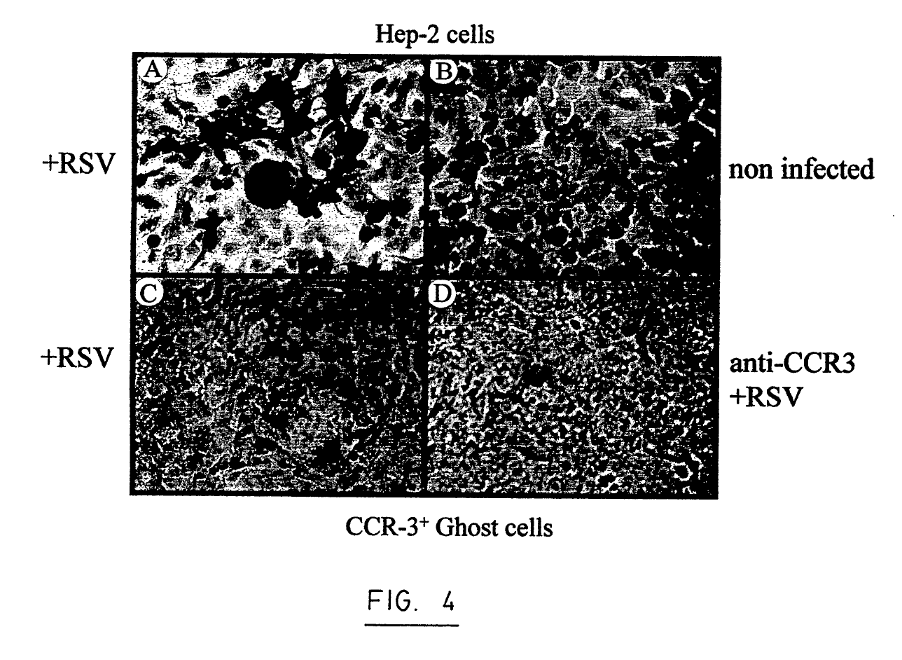

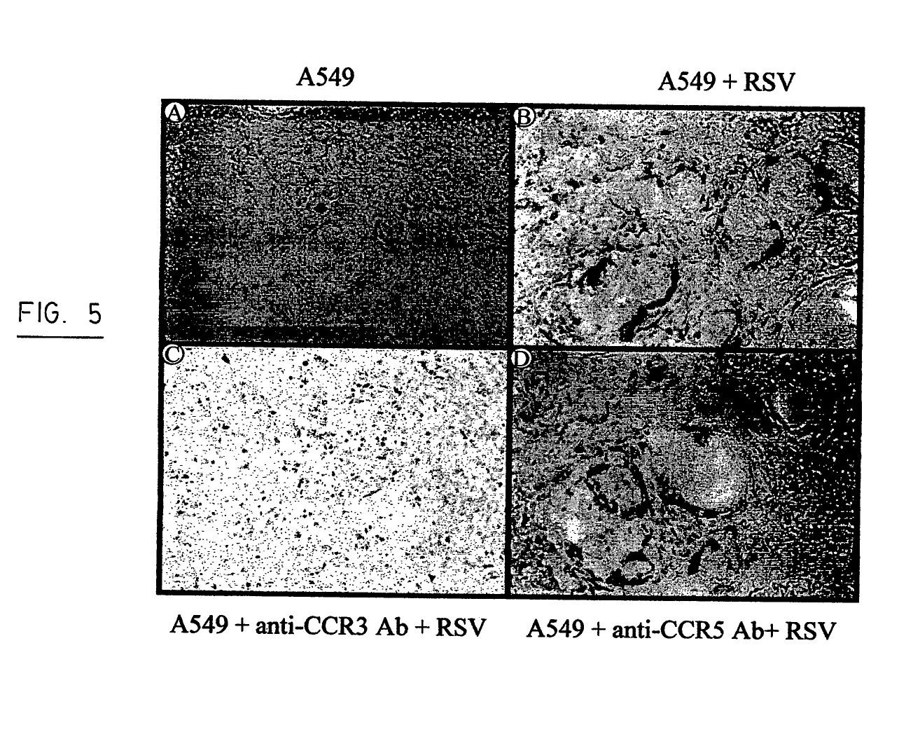

As detailed in the Exemplification section of this application, the RSV-G protein does bind to the CCR3 receptor (FIG. 1). Cells having the CCR3 receptor can be infected by RSV (FIG. 3 and FIG. 4) and soluble eotaxin (a ligand of CCR3 receptor) competes for virus binding to the cell CCR3 receptor in a dose dependent manner (FIG. 2 and FIG. 3). Similarly, neutralizing antibodies specific for the CCR3 receptor also inhibited RSV infection and replication (FIG. 4 and FIG. 5).

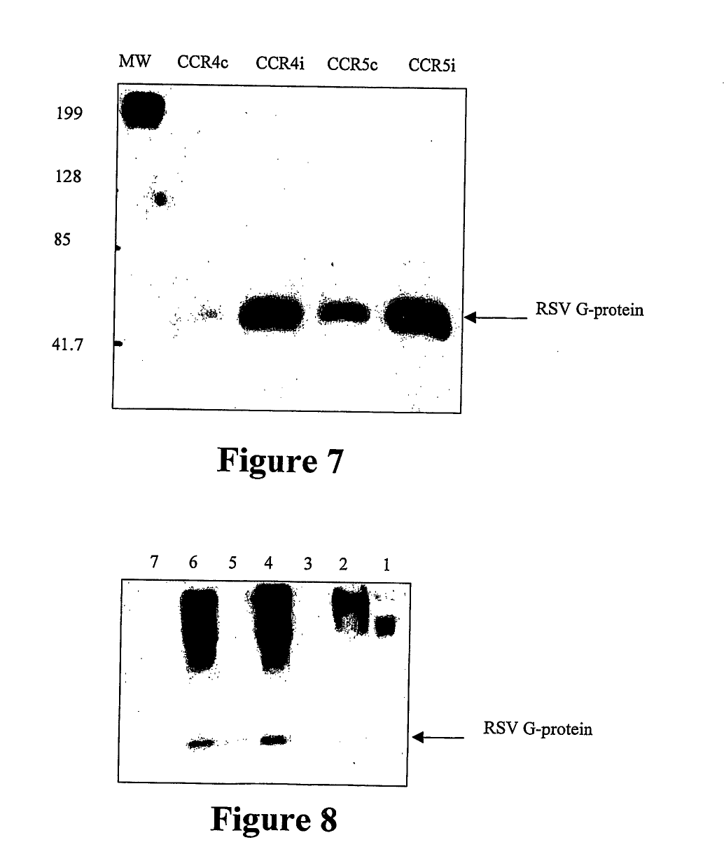

Interestingly, the present inventors found that the CCR1, CCR2, CCR4, CCR5 and CCR8 are also cellular coreceptors for RSV. As shown in FIGS. 7 and 8, GHOST cell lines expressing the chemokine receptors CCR1, CCR2, CCR4, or CCR5 support RSV infection whereas parental cells that do not express chemokine receptors are not infected. Homology studies (see hereinafter) permits to predict that CCR8 is most probably another RSV coreceptor.

It is to be understood however that the present application is not limited to the human RSV only. Indeed, the human RSV is a species of the genus Pneumovirus which includes the bovine respiratory syncytial virus (BRSV), the human respiratory syncytial virus (HRSV), the pneumonia virus of mice (PVM) and the turkey rhinotracheitis virus (TRTV). The genus Pneumovirus is itself a member of the Pneumovirinae subfamily which is a member of the larger family Paramyxoviridae. It is also recognized that the member viruses of the family Paramyxoviridae have a similar strategy of gene expression and replication and gene order to those of other families in the order Mononegavirales, that is the families Rhabdoviridae and Filoviridae (excerpted from the Universal Virus Database, http://life.anu.edu.au/viruses/lctv/index.html). Therefore, although it has not been exemplified herein, it is possible and even highly probable in certain cases, that CCR3 is a cellular coreceptor for other viruses from, or related to, the order Mononegavirales, and more particularly the family Paramyxovirdae, and even more particularly the Pneumovirinae subfamily. A person skilled in the art will be able to determine, without undue experimentation which viruses have surface proteins binding to the CCR1, CCR2, CCR3 CCR4, CCR5 and/or CCR8 and, identify whether these “CCR-binding” proteins, if any, are involved in the virus entry into host cells.

1) Brief Description of Human Chemokine Receptors

The chemokines are a super family of chemotactic cytokines that mediate leukocyte trafficking by binding to specific G protein linked seven transmembrane spanning receptors. Chemokines are divided into three groups based on the primary sequence of the first two cysteines: the C—X—C, C—C, and C families. Whereas the C—X—C and C families are mainly active towards neutrophils and lymphocytes, respectively, the C—C family members are active towards macrophages, lymphocytes, basophils, and eosinophils. The present inventors have found that several chemokine receptors are also present on epithelial cells, suggesting a role for chemokines in epithelial cells also. Although there are over 50 chemokines, it is highly likely that they originated from one common precursor and that there has been differentiation over time. It is also highly likely that certain homologies have been conserved in the primary, secondary and tertiary structures of chemokines and their receptors. These homologies can explain why a chemokine receptor like CCR3 has at least 6 ligands and why a ligand like RANTES that acts on CCR3 also acts on other chemokine receptors. In addition, it may not be necessary to have homologies in the primary structure to have homologies in the tertiary structure. Indeed, mammalian defensins have tertiary structure homologies with several chemokines and functional activity (acting through several chemokine receptors) even though chemokines and defensins have no apparent homology at the amino-acid level (De Yang et al. Trends in Immunology; 2002 vol 23, pp.291-296).

The chemokine receptor CCR3 was first described as encoded by a single gene that has 4 exons, the fourth exon is processed into a mature protein which becomes functional on the cell surface. Nucleotide sequences which encode CCR3 are known for human (GenBank accession Nos: AF262304, AF262303, AF262302, AF262301, AF262300, AF262299, AF247361 AF247360, AF247359, AB023887, AF224496, AF224497, AF224495, AF237380 and U51241), sheep (GenBank No: AF266468) macaque (GenBank Nos: AY065647, AY065646, AF291671, AF017283, Y13776, Y13775), cat (GenBank No: AF226606), rat (GenBank Nos: NM_{—}053958, AF003954), mouse (GenBank Nos: XM_{—}135270, NM_{—}009914) and guinea pig (GenBank No: AF060698). The CCR3 receptor is present on epithelial cells or on inflammatory cells such as basophils, lymphocytes and eosinophils, and it is part of the body's inflammatory response CCR3 is also described in details in international PCT patent applications WO99/66037, WO 97/41225, WO97/41154, and WO 96/22371. The human mRNA sequence (cDNA) for CCR3 is set forth herein as SEQ ID NO:5 and the corresponding predicted amino acid sequence is set forth herein as SEQ ID NO:5.

The CCR1 receptor is well known and is described in detail in many publications (see Murdoch and Finn, Blood, 2000, 95, 3032-3043 for a review). Nucleotide sequences which encode CCR1 are known for human (GenBank™ No: NM_{—}001293, NM_{—}001295, AF051305); mouse (GenBank™ No:NM_{—}009912); marmoset (GenBank™ No: AF127928); rabbit (GenBank™ No: AF127527) and macaque (GenBank™ No: AF017282). The human mRNA sequence (cDNA) of the CCR1 is set forth herein as SEQ ID NO:1, NM_{—}001295 and the corresponding predicted amino acid sequence is set forth herein as SEQ ID NO:2 NP_{—}001286. The receptor CCR1 is the high affinity RANTES (Regulated on T-cell Activation Normal T cell expressed and Secreted) and MIP-1a (Macrophage Inflammatory Protein) receptor. Although the chemokine MCP-3 (Monocytes Chemoattractant Protein) bind to CCR1 with moderate affinity, it is considered as a CCR1 ligand. IL-2 and IL-5 induces the expression of CCR1 on activated T cells, whereas II-10 selectively up regulates the expression of CCR1 on human monocytes.

The CCR2 receptor is well known and is described in detail in many publications (Murdoch and Finn, Blood, 2000, 95, 3032-3043 for a review). Nucleotide sequences which encode CCR2 are known for human (GenBank™ No: NM_{—}000647, NM_{—}000648, NM_{—}003965, U95626), rat (GenBank™ No:NM_{—}021866, U77349); mouse (GenBank™ No: XM_{—}125240), orangutan (GenBank™ No: AF354631); Gorilla (GenBank™ No: AF354630); macaque (GenBank™ No: AF124381). The human mRNA sequence (cDNA) of the CCR2 is set forth herein as SEQ ID NO:3, U95626, and the corresponding predicted amino acid sequence is set forth herein as SEQ ID No:4, NP_{—}000639. Both CCR2a and CCR2b have identical 5′ untranslated and transmembrane regions, but they differ in an alternatively spliced carboxyl terminus. Consequently the carboxy-tail of CCR2b is 36% homologous to the corresponding region in CCR1, whereas the carboxy tail of CCR2a bears no similarities to any other known chemokine receptor. MCP-1, 2, 3, 4, 5 are the CCR2b ligands. CCR2 expression in monocytes is decreased by both IFNγ and lopopolysaccharides, whereas IL-2 induces the expression in T lymphocytes.

The CCR4 receptor is well known and is described in detail in many publications (see Murdoch and Finn, Blood, 2000, 95, 3032-3043 for a review). Nucleotide sequences which encode CCR4 are known for human (GenBank™ No: AB023889, NM_{—}005508), mouse (GenBank™ No: XM_{—}135257, BG_{—}277031); rat (GenBank™ No: ) and zebrafish (GenBank™ No: ). The human mRNA sequence (cDNA) of CCR4 is set forth herein as SEQ ID NO:7, NM_{—}005508 and the corresponding predicted amino acid sequence is set forth herein as SEQ ID NO:8, P51679. The receptor CCR4 is highly expressed in Th2 cells and platelets and weakly expressed in other peripheral mononuclear cells. Although RANTES, MIP-1α and MCP-1 were reported as ligands for CCR4, TARC (Thymus and Activation Regulated chemokine) has been shown to be a selective chemoattractant ligand for CCR4 expressing cells.

The CCR5 receptor is well known and is described in detail in many publications (see Murdoch and Finn, Blood, 2000, 95, 3032-3043 for a review). Nucleotide sequences which encode CCR5 are known for human (GenBank™ No: AF161909 to AF161921, U54994, NM_{—}000579, AF082742), mouse (GenBank™ No: NM_{—}009917) and macaque (GenBank™ No: AF252565 to AF252568). The human mRNA sequence (cDNA) of CCR5 is set forth herein as SEQ ID NO:9, NM_{—}000579 and the corresponding predicted amino acid sequence is set forth herein as SEQ ID NO:10, AAB65737. The receptor CCR5 was found to bind the chemokine MIP-1β, RANTES, MIP-1α and MCP-2 specifically. CCR5 has been shown to be a major coreceptor in association with CD4 for macrophage-tropic (R5) HIV-1 strain entry to permissive cells.

The CCR6 receptor is well known and is described in detail in many publications (see Murdoch and Finn, Blood, 2000, 95, 3032-3043 for a review). Nucleotide sequences which encode CCR6 are known for human (GenBank™ No: XM_{—}033838, U45984), mouse (GenBank™ No:NM_{—}009835, AJ222714). The human mRNA sequence (cDNA) of CCR6 is set forth herein as SEQ ID NO 11, U45984, and the corresponding predicted amino acid sequence is set forth herein as SEQ ID NO:12, P51684. The receptor CCR6 has been detected on memory T cells, B lymphocytes, and dendritic cells but not any other peripheral blood leukocyte.

The CCR7 receptor is well known and is described in details in many publications (see Murdoch and Finn, Blood, 2000, 95, 3032-3043 for a review). Nucleotide sequences which encode CCR7 are known for human (GenBank™ No: XM_{—}049959, NM_{—}001838), mouse (GenBank™ No: XM_{—}122359) and rat (GenBank™ No: AF_{—}121670). The human mRNA sequence (cDNA) of CCR7 is set forth herein as SEQ ID NO:13, XM_{—}049959 and the corresponding predicted amino acid sequence is set forth herein as SEQ ID NO:14, NP_{—}001829. The receptor CCR7 is known to be expressed on activated T and B lymphocytes and dendritic cells and is strongly up-regulated in B cells infected with Epstein Barr virus and in T cells infected with herpesvirus 6 or 7. The receptor binds the chemokines ELC (EBI1-ligand chemokine) and SLC (secondary lymphoid tissue chemokine).

The CCR8 receptor is well known and is described in detail in many publications (see Murdoch and Finn, Blood, 2000, 95, 3032-3043 for a review). Nucleotide sequences which encode CCR8 are known for human (GenBank™ No: XM_{—}041048, AF005210, U45983) and mouse (GenBank™ No: NM_{—}007720, AF100201, Z98206). The human mRNA sequence (cDNA) of CCR8 is set forth herein as SEQ ID NO:15, U45983, and the corresponding predicted amino acid sequence is set forth herein as SEQ ID NO:16, NP_{—}005192. The CCR8 receptor bind the chemokine 1-309 which is a potent monocyte chemoattractant and bind specifically to CCR8 which is preferentially expressed on Th2 cells.

The CX3CR1 receptor is well known and is described in detail in many publications (see Murdoch and Finn, Blood, 2000, 95, 3032-3043 for a review). Nucleotide sequences which encode CX3CR1 are known for human (GenBank™ No: XM_{—}047502, NM 001337) and mouse (GenBank™ No:NM_{—}009987, XM_{—}147339, AF074912, AF0102269). The human mRNA sequence (cDNA) of CX3CR1 is set forth herein as SEQ ID NO:17, XM_{—}047502 and the corresponding predicted amino acid sequence is set forth herein as SEQ ID NO:18, NP_{—}001328. The CX3CR1 receptor is the natural receptor for fraktalkine which promotes adhesion of monocytes, NK cells and T lymphocytes to endothelial, epithelial and dendritic cells.

The CXCR4 receptor is well known and is described in detail in many publications (see Murdoch and Finn, Blood, 2000, 95, 3032-3043 for a review). Nucleotide sequences which encode CXCR4 are known for human (GenBank™ No: NM_{—}003467, AF025375, NM_{—}003467, AF348491, AF005058, AF052572), mouse (GenBank™ No: NM_{—}009911, Y14739), baboon (GenBank™ No: AF031089), cat (GenBank™ No: U63558) and macaque (U93311). The human mRNA sequence (cDNA) of CXCR4 is set forth herein as SEQ ID NO: 19, NM_{—}003467, and the corresponding predicted amino acid sequence is set forth herein as SEQ ID NO: 20, NP_{—}003458. The receptor CXCR4 is the natural receptor for SDF-1 (Stromal Derived Factor). This receptor was identified as an essential co-factor for T-tropic (X4) HIV-1 and HIV-2 type strains. SDF-1 is a highly efficacious lymphocyte chemoaftractant and it inhibits HIV-1 infection of permissive CD4+ cells. Mice lacking the CXCR4 gene exhibit impaired B lymphopoiesis, myelopoiesis, hematopoiesis, derailed cerebellar neurone migration, and defective formation of large vessels supplying the gastrointestinal tract.

2) Similarity Studies Between Chemokine Receptor Sequences

Given that it was recently demonstrated that the CX3CR1 facilitates HRSV infection of cells (Tripp et al. (2001) Nature Immunology, 8:732-738) and that the results presented in the Exemplification section of the present application clearly demonstrated that CCR3 was involved in HRSV entry into the cell, similarity studies (amino acid sequence alignment) of the primary structure were performed in order to find any consensus sequence(s) or region(s) that could be responsible for HRSV binding to both receptors. The sequence alignment also incorporated the sequence of other chemokine receptors in order to predict additional cell receptor(s) for HRSV.

Table 1 hereafter provides amino acid sequences identity for each of the CCR1, CCR2, CCR3, CCR4, CCR5, CCR6, CCR7, CCR8, CX3CR1 and CXCR4 human receptors. As can be appreciated, the CX3CR1 amino acid sequence shares the highest levels of identity and similarity with the CCR1, CCR2, CCR3, CCR4, CCR5, and CCR8 receptors. Interestingly, the CCR3 amino acid sequence also shares the highest levels of identity and similarity with the same receptors. These results strongly suggest that the CCR1, CCR2, CCR3, CCR4, CCR5, and CCR8 receptors could also have a HRSV binding activity, as does the CX3CR1 and CCR3.

TABLE 1
Comparison between human chemokine receptor amino acid sequences*.

| | CCR1 | CCR2 | CCR3 | CCR4 | CCR5 | CCR6 | CCR7 | CCR8 | CX3CR1 | CXCR4 |
|---|---|---|---|---|---|---|---|---|---|---|
| CCR1 | — | 56.6 | 63.2 | 53.1 | 57 | 36.3 | 39 | 44.3 | 43.7 | 33.3 |
| CCR2 | 56.6 | — | 52.3 | 48.2 | 75.9 | 37 | 40.5 | 42.8 | 45.8 | 34 |
| CCR3 | 63.2 | 52.3 | — | 49 | 53.3 | 37.6 | 38.1 | 38.3 | 42.6 | 32.4 |
| CCR4 | 53.1 | 48.2 | 49 | — | 50 | 41 | 38.6 | 46.9 | 44.7 | 39.7 |
| CCR5 | 57 | 75.9 | 53.3 | 50 | — | 36.1 | 38.7 | 42.5 | 44.6 | 34.8 |
| CCR6 | 36.3 | 37 | 37.6 | 41 | 36.1 | — | 38.7 | 42.5 | 36.8 | 33.9 |
| CCR7 | 39 | 40.5 | 38.1 | 50 | 38.7 | 38.7 | — | 31.3 | 37.4 | 35.3 |
| CCR8 | 44.3 | 42.8 | 38.3 | 46.9 | 42.5 | 42.5 | 31.3 | — | 41.5 | 34.4 |
| CX3CR1 | 43.7 | 45.8 | 42.6 | 44.7 | 44.6 | 36.8 | 37.4 | 41.5 | — | 34.8 |
| CXCR4 | 33.3 | 34 | 32.4 | 39.7 | 34.8 | 33.9 | 35.3 | 34.4 | 34.8 | — |

*Results are shown as percentage of identity

The present inventors have confirmed this hypothesis in vitro by assessing the infectivity of GHOST cells that are transfected with different chemokine receptors. As can be shown in FIGS. 7 and 8, GHOST cells that are transfected with either CCR1, CCR2b, CCR3, CCR4 or CCR5 produce glycosylated RSV-G protein after 72 hours of infection, whereas parental cells do not (not shown).

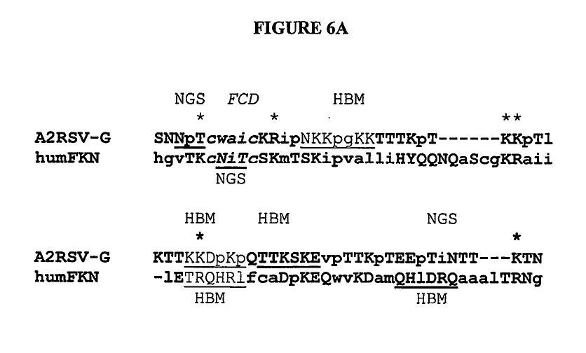

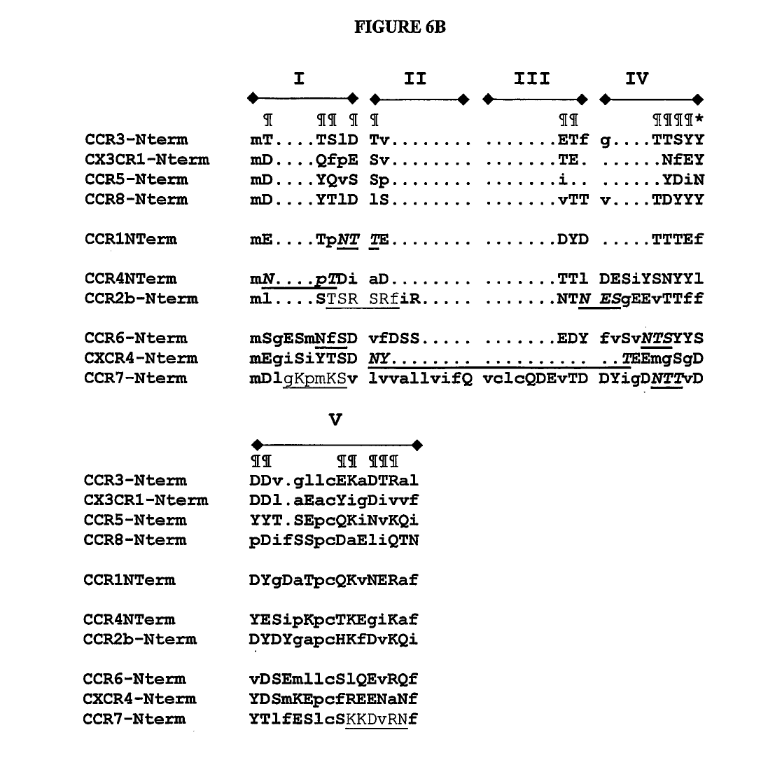

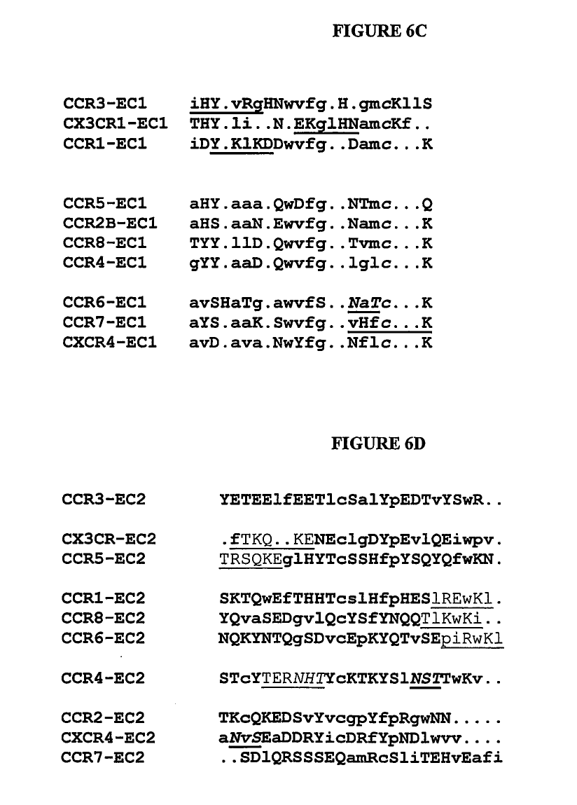

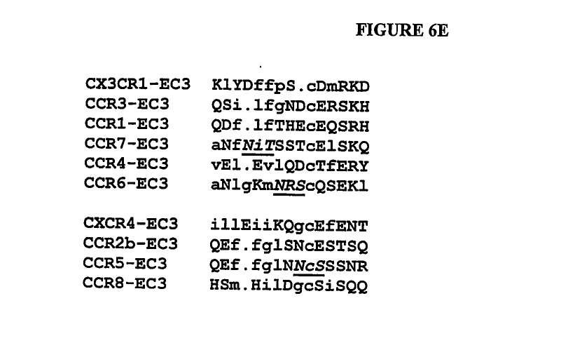

The present inventors have searched for consensus regions in the primary structure between CCR1, CCR2, CCR3, CCR4, CCR5 and CX3CR1. As can be shown in FIG. 6 there are many consensus regions in aminoacid sequences between these chemokine receptors. A large number are found in the intracellular/transmembrane domains (not shown). In the N-terminal region and the extracellular domain several consensus regions are also found. Interestingly amino acid similarity and conservative change are found in the N-terminal region and all 3 extracellular domains. When comparing CCR3 to CX3CR1 there is homology in the N-terminal region and in extracellular domains 1 and 3. Results of primary structure comparisaons would suggest that although all extracellular domains may participate in binding to HRSV, the N-terminal region and the first extracellular domain seem more important in the entry of HRSV into cells expressing this receptor.

HRSV has a propensity to infect epithelial cells. The present inventors have found that the chemokine receptors CCR3, CCR5, CXCR3 and CXCR4 are present on either the epithelial cell line A549 or on the epithelial cells present in biopsies of human airways. In addition, the chemokine receptors that the present inventors have found on epithelial cell lines are functional. Many other chemokine receptors have been reported on epithelial cells by other groups. As previously mentioned for defensins (De Yang et al. Trends in Immunology; 2002 vol 23, pp. 291-296), it is possible that the homology between RSV and its complementary structure on chemokine receptors is sufficient to cause functional effects not only on epithelial cells but also on inflammatory cells (and thus induce chemotaxis and activation of inflammatory cells). This hypothesis could explain a major finding that has been described in HRSV infection. Indeed, bronchiolitis is a common infection of the airways of infants that is mostly caused by HRSV and described histologically as an inflammatory cell influx into the small airways or bronchioles. It is generally thought that the immune response to HRSV leads to the recruitment of inflammatory cells into the bronchioles. However, the findings presented in this application would suggest that HRSV is directly involved in inducing the recruitment of inflammatory cells into the bronchioles through homologies with the CCR1, CCR2, CCR3, CCR4, CCR5 and CCR8 receptors. Results presented in FIGS. 2 and 3 showing that eotaxin can inhibit RSV infection of cells bearing CCR3 receptors would suggest that HRSV attaches to the CCR3 receptor at a site that has functional effects in cells. Interestingly, the CCR3 receptor is mostly involved in eosinophil chemotaxis and eosinophils are a predominant inflammatory cell in the airways of infants with bronchiolitis.

Knowledge of the primary and tertiary consensus sequences between the CCR1, CCR2, CCR3, CCR4, CCR5 and CCR8 receptors can help one create substances to block or treat RSV infection by designing competitors or inhibitors of RSV attachment to its coreceptor. In addition, these substances could have broad anti-inflammatory potential by potentially having inhibitory effects on several chemokine receptors. Knowledge of the tertiary structure of the coreceptor and its epitope on RSV could also be employed in the design of novel vaccination strategies.

C) Methods, Reagents and Compositions for Modulating Viral Infection of Cells

In view of the above, one aspect of the invention relates to a method for modulating viral infection of a cell. The method comprises modulating a binding interaction between a cell chemokine-receptor and a surface protein of the virus. The cell chemokine-receptor comprises an amino acid sequence encoded by a nucleic acid having a sequence at least 75%, 85%, or 95% identical to nucleotides 4015 to 5082 of SEQ ID NO:5 (CCR3). More preferably, the cell chemokine-receptor comprises an amino acid sequence having at least 50%, 65%, 75%, 85%, 90% or 95% similarity with SEQ ID NO:6 (CCR3), and even more preferably, at least 38%, 40%, 50%, 60%, 65%, 75%, 85%, 90% or 95% identity with SEQ ID NO:6 (CCR3). In a specific embodiment, the cell chemokine-receptor consists of CCR1, CCR2, CCR3, CCR4, CCR5, and/or CCR8.

A most specific aspect of the invention concerns a method for modulating a pneumovirus infection of a cell, comprising modulating a binding interaction between a cell chemokine-receptor and a surface protein. More preferably, the cell chemokine-receptor consists of CCR1, CCR2, CCR3, CCR4, CCR5, and/or CCR8. Therefore, a further aspect of the invention relates to a method for reducing pneumoviral infection of a CCR1-, CCR2-, CCR3-, CCR4-, CCR5-, and/or CCR8-positive cell, comprising interfering with binding of a pneumovirus to CCR1, CCR2, CCR3, CCR4, CCR5, and/or CCR8 receptor(s) of the cell.

According to a specific aspect of the invention, the viral infection is reduced or blocked by inhibiting or blocking the binding interaction that may occur between the cell receptor and the surface protein of the virus. As described hereinafter, this may be achieved by using different means. Accordingly, specific aspects of the invention concern methods of attenuating the ability of a pneumovirus to bind a mammalian cell, methods for pneumovirus prophylaxis in a mammal at risk, methods for reducing infectivity of Pneumoviruses, and methods for reducing the initiation or spread of a respiratory tract disease due to human RSV.

1) CCR-Ligands

One method of attenuating the ability of virus to bind cells is to expose the cells to a CCR-ligand that recognizes the CCR receptor under conditions sufficient for the ligand to bind the receptor. The bound CCR-ligand interferes with subsequent interaction between the virus and the receptor. Therefore, by such treatment, the ability of the virus to subsequently bind and infect the cells is attenuated and even blocked.

In a preferred embodiment, the CCR-ligand attenuates and more preferably blocks the ability of HRSV to bind CCR3 positive-cells, and more particularly human epithelial cells and human inflammatory cells such as basophils, lymphocytes and eosinophils and possibly cells of other mammals.

For example, for cells cultured in vitro, the CCR ligand can be added to the cell culture for a time sufficient for it to bind the corresponding CCR receptor(s) on the cells. For cells in vivo, the CCR ligand can be supplied in a pharmacologically acceptable carrier (e.g., a solution, gel, magma, or salve). In this regard, the CCR ligand can be delivered topically to cells within a discrete organ or tissue or systemically to attenuate a viral infection on a more wide-spread scale. The CCR ligand is exposed to the cells under conditions sufficient so that it binds the cells. The concentration of the CCR ligand, as well as the conditions required for efficient binding, will depend on the type of ligand employed, the type of receptor(s) targeted and location of delivery. However, it is within the routine skill in the art to investigate the kinetic profile of such ligands in advance of an application. The CCR-ligand may be selected from the ligands given hereinafter for the CCR1, CCR2, CCR3, CCR4, CCR5, and CCR8 receptors.

In this context, the CCR1—, CCR2-, CCR3-, CCR4-, CCR5-, and CCR8-ligand is present on any molecule suitable for blocking the interaction between the virus and the corresponding CCR receptor.

Well known CCR1 ligands include: regulated on activation normal T-cell expressed and secreted (RANTES); macrophage inflammatory protein-1α (MIP-1α) and MCP-3. Well known CCR2 ligands include: monocyte chemoattractant protein-1 (MCP-1), MCP-2, MCP-3, MCP-4, and MCP-5. Well known CCR3 ligands include: eotaxin 1, eotaxin 2 or eotaxin 3, MCP-3 and MCP4, and RANTES. Well known CCR4 ligands include MCP-1, RANTES, MIP-1α and Thymus and Activation Regulated chemokine (TARC). Well known CCR5 ligands include: MIP-1α, MIP-1β and RANTES. A well known CCR8 ligand is 1-309. The above list of CCR-ligands is not exaustive and any other suitable CCR-ligand could also be used according to the present invention.

While any molecules chemical or biological, can serve as the CCR-ligand or provide the CCR-ligand, the CCR-ligand is generally present as part of a protein. For example, the CCR-ligand can be an antibody recognizing an epitope on the CCR1, CCR2, CCR3, CCR4, CCR5, and/or CCR8 receptor. In other embodiments, the CCR-ligand is on a protein including an external domain of a virus envelope (e.g., RSV-G, RSV-F, or a soluble functional derivative or fragment of RSV-G or RSV-F capable of binding the CCR1, CCR2, CCR3, CCR4, CCR5, and/or CCR8 receptor). However, any compound which can bind to the CCR1, CCR2, CCR3, CCR4, CCR5, and/or CCR8 receptor can be considered as a CCR-ligand, including, without limitation, any chemicals, nucleic acids, peptides, proteins, glycoproteins, chemokines hormones, receptors, antibodies, antigens, nucleic acids, carbohydrates, polysaccharides, lipids, pathogens, virus, chemical substances, inhibitors, cofactors, substrates, growth factors, metabolites, analogs, drugs, dyes, mimetic molecules thereof.

In the preferred embodiments exemplified hereinafter, the CCR3 ligands are soluble proteins such as eotaxin 1 and neutralizing antibodies specific for the CCR3 receptor. In preferred embodiments, the CCR1 ligands are RANTES and neutralizing antibodies specific for the CCR1 receptor. Preferred CCR2 ligands include the MCP's and neutralizing antibodies specific for the CCR2 receptor. In preferred embodiments, the CCR4 ligands are MIP-1β and neutralizing antibodies specific for the CCR4 receptor. In preferred embodiments, the CCR5 ligands are MIP-1β and neutralizing antibodies specific for the CCR5 receptor. In preferred embodiments, the CCR8 ligands are 1-309 and neutralizing antibodies specific for the CCR8 receptor.

Other preferred CCR-ligands includes soluble viral surface molecules, fragments and analogues thereof and any other molecule that would compete with virus binding (preferably HRSV) to the cell chemokine receptor and thereby prevent, inhibit, or treat viral infections and more particularly HRSV related diseases such as broncholititis, bronchitis, pneumonia and asthma.

2) Virus-Ligands

Another approach of attenuating the ability of the virus to bind cells is to expose the virus, or a portion thereof, to a virus-ligand that recognizes viral cell surface molecule(s) under conditions sufficient for the virus ligand to bind the viral cell surface molecule(s) so that the bound ligand interferes with subsequent interaction between the virus and the CCR receptor. Thus, by such treatment, the ability of the virus to subsequently bind cells is attenuated and even blocked. Although any viral cell-surface molecule can be targeted, the virus ligand preferably recognizes the viral proteins that bind the cellular CCR1, CCR2, CCR3, CCR4, CCR5, and/or CCR8 receptors.

In this context, the virus-ligand consists of (or is present on) any molecule that interacts with viral cell-surface molecules and that is suitable for interfering or blocking the interaction between the virus cell-surface molecule(s) and the CCR1, CCR2, CCR3, CCR4, CCR5, and/or CCR8 receptors of a cell or a functional fragment or analog of the CCR1, CCR2, CCR3, CCR4, CCR5, and/or CCR8. As for the CCR-ligand defined hereinabove, any synthetic or natural substance or molecules can serve as the virus-ligand, and its methods of use are similar to those of the CCR-ligand. For instance, to a certain extent, a CCR3 virus-ligand may be a CCR3-positive cell or an animal having CCR3-positive cells.

According to the present invention, preferred virus ligands include monoclonal or polyclonal antibodies and fragments or functional analogs thereof that are capable of binding viral cell-surface molecules, and more preferably to the HRSV-G or HRSV-F proteins.

Other preferred virus ligands include soluble CCR1, CCR2, CCR3, CCR4, CCR5, and/or CCR8 receptors or fragments thereof and any other molecules or drugs that would mimic or compete with the normal cell CCR receptor(s) and thereby prevent, inhibit, or treat infection and more particularly HRSV related diseases such as broncholititis, bronchitis, pneumonia and asthma.

3) Modulation of Chemokine Receptor(s) Cellular Levels

Up to date, a powerful pharmaceutical strategy to prevent viral infection has been to target the viral receptors of the cell with pharmaceutically active compounds in order to inhibit their function, synthesis, or expression (see for example patents WO 00142300 for the Herpes virus receptor, and EP0414035 for Gibbon ape leukemia virus receptor).

Therefore, according to another aspect of the invention, it is provided a method for inhibiting the expression a virus cellular receptor, preferably a paramyxovirus receptor, more preferably a pneumovirus receptor and even more preferably a human RSV receptor, so that the number of receptor molecules per cell is reduced, thereby limiting the virus ability to bind and infect the cell and/or a host.

Many compounds and methods may be used to inhibit/reduce the expression cellular receptors. Examples of compounds include receptor polypeptides, fusion proteins, antigenic peptides, anti-receptor antibodies, peptidomimetics of these, and antisense oligonucleotides complementary to the receptor mRNA.

According to a preferred embodiment, the methods/compounds inhibit the function, synthesis, expression, or number, of the CCR1, CCR2, CCR3, CCR4, CCR5, and/or CCR8 receptors per cell. Suitable compounds are chosen among the group of molecules specific for and inhibitory of the CCR receptor(s), its corresponding protein precursors, or its corresponding nucleic acids (RNA, mRNA or DNA gene). These include:
(a)antibodies, CCR-ligands, and other compounds that bind to the CCR1, CCR2, CCR3, CCR4, CCR5, and/or CCR8 receptors; and
(b) antisense oligonucleotides, ribozymes, and other compounds that bind to CCR1, CCR2, CCR3, CCR4, CCR5, and/or CCR8 gene sequence, RNA or mRNA. For the CCR3, preferred antisense oligonucleotides include those described in WO 99/66037 which is incorporated herein.

These compounds are preferably incorporated into a pharmaceutical composition which is administered to an uninfected person or animal susceptible to viral infection, or to a patient infected with the virus, so as to treat, inhibit or prevent the viral infections and more particularly HRSV related diseases such as broncholititis, bronchitis, pneumonia and asthma. For CCR antisense molecules, these may be introduced or expressed into the cell by using any suitable means known to those skilled in the art.

According to a preferred embodiment, expression of the CCR1, CCR2, CCR3, CCR4, CCR5, and/or CCR8 is reduced and even silenced, so that the ability of HRSV to bind and infect cells is diminished and even totally blocked.

Therefore, further aspects of the invention concern methods of attenuating the ability of a pneumovirus to bind a mammalian cell, methods for pneumovirus prophylaxis in a mammal at risk, methods for reducing infectivity of Pneumoviruses, and methods for reducing the initiation or spread of a respiratory tract disease due to human RSV. Essentially, these methods are based on the use of CCR-ligands, virus-ligands or antisense molecules (preferably incorporated into effective therapeutic compositions) for attenuating and even blocking the ability of a pneumovirus such as the HRSV to bind and infect cells.

The method of attenuating the ability of a pneumovirus to bind a mammalian cell, preferably comprises a step consisting of:
exposing the cell to a CCR-ligand that recognizes at least one CCR1, CCR2, CCR3, CCR4, CCR5, and CCR8 chemokine-receptor, the exposition being carried out under conditions sufficient for the CCR-ligand to bind the cell receptor(s);
exposing the virus to a virus-ligand that recognizes a surface protein of the pneumovirus, the exposition being carried out under conditions sufficient for said virus-ligand to bind said pneumovirus surface protein and subsequently attenuate binding of pneumovirus to at least one of the CCR1, CCR2, CCR3, CCR4, CCR5, and/or CCR8 chemokine-receptors; and/or
reducing intracellular levels of the CCR1, CCR2, CCR3, CCR4, CCR5, and/or CCR8 chemokine-receptors; thereby limiting the pneumovirus ability to bind the mammalian cell.

The method for attenuating the ability of a pneumovirus to infect a mammalian cell expressing a CCR1, CCR2, CCR3, CCR4, CCR5, and/or CCR8 chemokine-receptor(s) comprises exposing the cell to a CCR-ligand that recognizes at least one of the cell chemokine-receptor, the exposure being carried out under conditions sufficient for the CCR-ligand to bind said at least one of the chemokine-receptors, whereby the ability of said pneumovirus to subsequently infect said cell is attenuated.

The method for pneumovirus prophylaxis in a vertebrate at risk, comprises:
a) providing a composition comprising a virus-ligand exhibiting the ability to bind to a surface protein of the pneumovirus (the surface protein being involved in binding of the pneumovirus to a CCR1, CCR2, CCR3, CCR4, CCR5, and/or CCR8 receptors); and
b) administering the composition to the vertebrate at risk.

The method for reducing infectivity of Pneumoviruses comprises contacting the virus under conditions favorable for binding with a virus-ligand, the virus-ligand exhibiting the ability to bind to a viral surface protein involved in binding of the pneumovirus to a CCR1, CCR2, CCR3, CCR4, CCR5, and/or CCR8 chemokine-receptor.

The method for reducing the initiation or spread of a respiratory tract disease due to human RSV, comprises administering to a human an antiviral agent comprising a virus-ligand which exhibits the ability to bind to HRSV and reduce infectivity thereof, the virus-ligand exhibiting the ability to bind to a HRSV surface protein involved in binding of the HRSV to a CCR1, CCR2, CCR3, CCR4, CCR5, and/or CCR8 cell chemokine-receptor.

As mentioned previously, pneumovirus ability to infect cells may be limited by reducing the intracellular levels of the CCR receptor(s) by introducing or expressing into the cell CCR antisense oligonucleotides. According to a preferred embodiment, the antisense oligonucleotide is administered, directly to the respiratory system. Preferably, the oligonucleotides of the invention would be incorporated into a pharmaceutical composition comprising at least one of the oligonucleotides defined previously, and a pharmaceutically acceptable carrier.

The amount of oligonucleotides present in the composition of the present invention is a therapeutically effective amount. A therapeutically effective amount of oligonucleotides is that amount necessary so that the oligonucleotide perform its biological function without causing overly negative effects into the host to which the composition is administered. The exact amount of oligonucleotides to be used and composition to be administered will vary according to factors such as the oligonucleotides' biological activity, the type of condition being treated, the mode of administration, as well as the other ingredients in the composition. Typically, the composition will be composed from about 1% to about 90% of oligonucleotide(s), and about 20 μg to about 20 mg of oligonucleotide will be administered. For preparing and administering such pharmaceutical compositions, methods well known in the art may be used.

4) Stimulation of the Host Defense Mechanisms

Another method of attenuating the ability of viruses such as human RSV to bind cells is to induce/stimulate a host immunological response against viral cell-surface molecule(s), preferably against viral CCR binding proteins, so that the host immunological response interferes with the virus ability to interact with the CCR receptor(s).

Accordingly, a host that is infected or susceptible to be infected could be immunized against viral cell-surface molecules so that it develops an immunological response that will interfere with the virus' ability to interact with the CCR1, CCR2, CCR3, CCR4, CCR5, and/or CCR8 receptors. For instance, it is known that for HRSV, the cell mediated response to viral infection in humans produces cytotoxic T cells that recognize the RSV F protein, matrix (M) protein, SH, and the nonstructural protein 1b.

Therefore, the present invention also relates to the use of viral surface proteins (such as HRSV-G protein) fragments and analogs thereof for inducing/stimulating a host immunological response, the immunological response interfering with the virus ability to interact with the CCR1, CCR2, CCR3, CCR4, CCR5, and/or CCR8 receptors and infect such CCR-positive cells.

Methods for inducing an immunological response are well known in the art, and typically consist of administering to the host viral antigens or epitopes with or without carriers, adjuvants or immune modulators against which an immune response is to be raised. The viral antigens may be purified (see U.S. patent application Ser. No. 08/679,060 and international PCT application WO 9801257 which give examples for the preparation of RSV proteins) or prepared using chemical or recombinant techniques. Similarly, animals could possibly be inoculated with vaccinia or other virus recombinants expressing RSV-G or —F antigens. A person skilled in the art will know how to select and use suitable antigens in order to induce/stimulate an effective host immunological response against viral cell-surface molecule(s).

5) Pharmaceutical Compositions and Vaccines

According to a further related specific aspect, the present invention provides pharmaceutical compositions and vaccines which attenuate and more preferably block the ability of viruses such as HRSV to bind CCR1-, CCR2-, CCR3-, CCR4-, CCR5-, and/or CCR8-positive cells. Preferred compositions are those intended for treating and/or preventing infection by a pneumovirus.

According to a preferred embodiment, the compositions/vaccines of the invention comprise a i) CCR-ligand and ii) a pharmaceutically acceptable carrier. The CCR-ligand exhibits the ability to bind to at least one of the CCR1, CCR2, CCR3, CCR4, CCR5, and CCR8 chemokine-receptor, and thereby reducing infectivity of the pneumovirus.

According to another preferred embodiment, the compositions/vaccines of the invention comprise i) a virus-ligand that recognizes cell-surface molecule(s) of the virus, preferably viral CCR-binding proteins, under conditions sufficient for the viral ligand to bind viral cell-surface molecule(s) and interfere with the virus ability to interact with the CCR receptor, and ii) a pharmaceutical acceptable carrier.

According to a third aspect, the compositions/vaccines of the invention comprise I) a compound, preferably a viral antigen, that induces/stimulates a host immunological response against cell-surface molecule(s) of the virus, preferably viral CCR-binding proteins, so that the host immunological response interfere with the virus ability to interact with the CCR receptor, and ii) a pharmaceutical acceptable carrier.

Immunogenic compositions or vaccines according to the present invention may be prepared as injectables, or as liquids (solutions, suspensions, or emulsions) or as oral tablets, pills, capsules, sustained release formulations or powders, suppositories, or skin patches, containing 0.1% to 100% vol/vol of the active ingredients. The immunogenic ingredients may be mixed with pharmaceutically acceptable excipients such as saline, water, alcohols, sugars, wetting and emulsifying agents, liposomes, pH buffering agents, and adjuvants. Immunogenic compositions or vaccines may be administered parenterally by injection (intramuscular, subcutaneous, intradermal); by spraying onto mucosal surfaces in the nose, mouth or into the lower airways or taken orally (swallowed). For administration by suppositories, binders and carriers may be included such as glycols or triglycerides. Oral formulations may include carriers such as cellulose, saccharine, magnesium carbonate. More preferably, the composition consists of a spray for application to airway tissues susceptible to infection by HRSV.

Immunogenicity can be significantly enhanced when the active immunogenic ingredients of a vaccine are mixed with an adjuvant. Adjuvants act by retaining the antigens locally where administered, thus causing a sustained release of the antigens to the immune system. They can also attract immune cells to the antigens and stimulate the immune cells to respond to the antigens. Typical adjuvants for human vaccines include alum (aluminum hydroxide and aluminum phosphate). Other ways of improving the immunogenicity of a vaccine include the use of more specific immunomodulators such as CpG oligonucleotides, antisense oligonucleotides, DNA, gene therapy that can be linked directly to the vaccine or administered independently.

The amount of active ingredients present in the composition of the present invention is a therapeutically effective amount. A therapeutically effective amount of active ingredients is that amount necessary so that the active ingredients perform their desired biological function without causing overly negative effects into the host to which the composition is administered. The effective doses of immunogenic preparations and vaccines typically vary with the route of administration and the age, weight, and medical condition of the individual patient. Suitable doses are readily determined by one skilled in the art and are typically within the range of micrograms to milligrams of each of the active ingredients. The dosing schedule or regime for initial administration and booster doses are readily determined by one skilled in the art and also depend on the above described variables.

D) Methods and Kits for the Diagnostic or Prognostic of a Viral Infection

According to another aspect of the invention, it is provided methods and kits for the diagnostic or prognostic of a viral infection, such as an infection caused by a pneumovirus and more particularly an infection by the Human Respiratory Syncytial Virus (HRSV).

More particularly, the invention provides a method for detecting the presence of a pneumovirus (preferably HRSV) in a biological sample, e.g. mucosal, buccal, nasal, airway secretions, ocular secretions, cerebrospinal fluid, serum or blood. According to an embodiment, the method involves:
a) contacting the biological sample with a cell expressing at least one of the CCR1, CCR2, CCR3, CCR4, CCR5, and CCR8 chemokine-receptors, the contacting step being carried out under conditions sufficient for pneumovirus(es) contained in the sample, in any, to infect the cell;
b) detecting the presence of pneumovirus(es) in the cell.

A person skilled in the art will easily determine what is the best type of cells to use. Examples of CCR3-positive cells that may be used includes Hep-2 cells, A549 cells, GHOST cells transfected with the CCR3 receptor. After a certain period of time (from 2 to 48 hours) such cells would contain thousands of copies of the RSV virus. A non limitative list of methods for detecting the presence of RSV in infected cells includes direct visualization, microscopic visualization, immunostaining of the cells, in situ hybridization, PCR or sequencing analysis on viral genetic material.

If necessary, the cell may be pretreated with agent(s) capable of increasing or permitting the expression of the chemokine-receptor(s) on the cell surface (for instance, GHOST cells may also be transfected with any of the CCR1, CCR2, CCR3, CCR4, CCR5 and/or CCR8 receptor(s)). Increasing the expression of CCR receptor(s) on the cell surface may increase the sensitivity of the cells to pneumovirus infection such that the presence of the pneumovirus is detected in the biological sample more quickly or more often. Preferred agents for increasing the expression of CCR receptor(s) within a cell are cell stimulants or activators found within but not limited to the following groups: mediators, cytokines, chemokines, lectins, proteins, proteoglycans, and ions.

Another method for detecting the presence of Pneumoviruses in a biological sample involves contacting the biological sample with a compound, an agent and even a cell, capable of binding a surface molecule of the pneumovirus such that the presence of pneumovirus is detected in the biological sample. Preferred agent for binding Pneumoviruses surface molecule(s) includes virus-ligands as defined hereinbefore. The ligands may be fixed or adsorbed covalently or not to a support. The support may be made of natural or synthetic molecules. Well known synthetic macromolecular support includes polylysine and poly(D-poly(D-L-alanine)-poly(L-lysine). Other types of suitable support include liposomes, microparticles, microspheres made of latex, polyphosphoglycans (PGLA), or polystyrene.

A prognostic or diagnostic kit according to the invention would preferably comprise a compound, an agent or a cell capable of binding a pneumovirus surface molecule, and instructions, assay tubes, enzymes, or reagents such that the binding of RSV surface molecule be detected. Preferred compounds or agents for binding a RSV surface molecule include virus-ligands as defined hereinbefore. These virus-ligands may be fixed or adsorbed covalently or not to a support as described previously.

The methods and kits for the diagnostic or prognostic of viral infections according to the invention may be very useful to detect human or animal infection on specimens obtained from potentially infected fluids, secretions or tissue, to increase the speed, sensitivity and specificity of assays, and to help in the management of pneumoviral infections and/or related diseases such as bronchiolitis, bronchitis, pneumonia, and asthma.

E) Screening Methods and Kits

According to another aspect, the invention relates methods and kits for screening, identifying and/or evaluating new antiviral compounds (e.g. chemical, drugs, vaccine, etc) that are capable of interfering with a viral binding interaction with the cell CCR1, CCR2, CCR3, CCR4, CCR5, and/or CCR8 receptors. These antiviral compounds could be used for inhibiting, prevention and/o reducing viral infection of cells by a virus of the order Mononegavirales, and more particularly CCR1-, CCR2-, CCR3-, CCR4-, CCR5-, and/or CCR8-positive cells.

In a preferred embodiment, the method comprises the steps of:
contacting a functional virus-ligand (as defined herein before) and a surface protein of a virus in presence of a potential antiviral compound, the surface protein of the virus being involved in binding of the virus to a CCR1, CCR2, CCR3, CCR4, CCR5, and/or CCR8 receptor expressed by the cell;
measuring a binding interaction between the virus-ligand and the surface protein of the virus; whereby an antiviral compound is selected when said binding interaction is measurably reduced in presence of the potential antiviral compound.

According to another embodiment, the method of the invention consists of an in vitro method for selecting an antiviral compound that is capable of reducing a pneumoviral infection of a cell expressing a CCR1, CCR2, CCR3, CCR4, CCR5, and/or CCR8 receptor. The method comprises the steps of:
a) providing a test tube comprising: (i) a functional CCR-ligand of at least one the chemokine-receptors, (ii) a compound or a cell comprising a virus-ligand, and (iii) at least one potential antiviral compound;
b) measuring a binding interaction between the virus-ligand and the functional CCR-ligand; and
c) comparing the measure obtained with a control value; whereby an antiviral compound is selected when the binding interaction is measurably reduced as compared to the control value.

According to a further embodiment, the method of the invention comprises the steps of:
a)providing a host cell expressing at least one functional CCR1, CCR2, CCR3, CCR4, CCR5, and/or CCR8 receptor;
b)providing a functional ligand for the receptor(s) defined in (a);
c)contacting a potential antiviral compound with the cell; and
d)measuring a binding interaction between the functional receptor(s) and the functional ligand; whereby an antiviral compound is selected when the binding interaction is measurably reduced as compared to a control value.

Of course, reference to a “measurably reduced binding interaction” and “as compared to a control value” imply that the results obtained are compared indirectly or directly to some type of experiment wherein the method is carried out in absence of the antiviral compound(s) to be tested. However this is not a prerequisite since a “reference value” such as a control value could be obtained by any other means or simply provided or incorporated into a suitable automatic device for carrying out the methods.

Another related aspect of the invention concerns a kit for selecting an antiviral compound that is capable of reducing a pneumoviral infection of a CCR1-, CCR2-, CCR3-, CCR4-, CCR5-, and/or CCR8-positive cells. The kit of the invention comprises: i) a functional CCR-ligand of the cell receptor(s), and ii) a compound or a cell comprising a virus-ligand; the binding of the functional CCR-ligand with the virus-ligand being assayable.

Preferred compounds or agents comprising a functional CCR-ligand include those defined hereinbefore. Preferably, the CCR-ligand is the G-protein of the HRSV or a functional fragment or analog thereof. The CCR-ligand may also be provided by means of viruses or cells having surface molecules capable of binding the CCR1, CCR2, CCR3, CCR4, CCR5, and/or CCR8 receptors.

Preferred virus-ligands include those defined hereinbefore. These virus-ligands may be fixed or adsorbed covalently or not to a support as described previously. They may also be provided by means of cells or animals expressing the CCR1, CCR2, CCR3, CCR4, CCR5, and/or CCR8 receptors.

Depending on the nature of the antiviral compounds to be tested, the virus-ligands and/or the CCR-ligand, the binding interaction may be measured using well known methods including but not limited to enzyme-linked immunosorbent assays (ELISA), filter binding assays, FRET assays, scintillation proximity assays, microscopic visualization, immunostaining of the cells, in siftu hybridization, PCR or sequencing analysis on viral genetic material.

An assay kit according to the invention would preferably further comprise instructions, assay tubes, enzymes, reagents or cells, such that the binding of the virus-ligands to the CCR-ligand (or vice-versa) being assayed or detected.

The screening methods and kits according to the invention may be very useful to screen and evaluate new compounds, drugs and vaccines that are capable of interfering with virus binding interaction with the CCR receptors and thereby discover new medicines effective against various viruses (such Pneumoviruses) and effective for treating/preventing viral infections, bronchiolitis, bronchitis, pneumonia, and asthma, and more particularly infections by the Human Respiratory Syncytial Virus (HRSV). Therefore, the present invention encompasses all antiviral compounds identifiable via any one of the methods or kit defined previously. This same screening method could also be applicable to the search for novel anti-inflammatory compounds capable of inhibiting the binding of ligands to several chemokine receptors (CCR1, CCR2, CCR3, CCR4, CCR5 and CCR8).

F) Cells and Transgenic Animals with Increased or with Lowered Risk of Viral Infection

According to another aspect, the invention relates to the use of cells and transgenic animals with increased or lowered risk of viral infection. Indeed, since it has been found that the CCR1, CCR2, CCR3, CCR4, CCR5, and CCR8 receptors are cell coreceptors for RSV, it may be useful to modulate levels of these chemokine receptors in cells and animals in order to modify accordingly the susceptibility of these cells/animals to a virus infection.

For instance, cells or animals with lower or higher levels of CCR1, CCR2, CCR3, CCR4, CCR5, and/or CCR8 could be used to: i) study in vitro, ex vivo or in vivo the mechanisms of viruses infection, ii) to screen and evaluate new compounds, vaccines and drugs that are capable of interfering with viruses binding interaction with the CCR receptors, iii) to confirm that the infecting virus is indeed a specific virus (e.g. RSV) by using cells that either do or do not express given CCR receptor(s).

Also, non human transgenic animals with reduced levels of the CCR receptors, or having no CCR receptors at all, would be much less sensitive to virus infections and more particularly Pneumoviruses infections, an important economic advantage, particularly for domestic and farm animals. Accordingly, the present invention encompasses genetically modified cells and animals with increased or with lowered risk of viral infection.

Therefore, the invention provides a method for producing a cell or a non-human animal having a modified risk of viral infection. The method comprises the step of modifying cellular expression levels of at least one CCR1, CCR2, CCR3, CCR4, CCR5, and/or CCR8 chemokine-receptor, thereby modifying accordingly susceptibility of the cell or non-human animal to a viral infection.

Another specific aspect of the invention concerns a method for increasing viral infection of a cell. The method comprises the step of permitting or increasing a binding interaction between at least one CCR1, CCR2, CCR3, CCR4, CCR5, and/or CCR8 chemokine-receptor of the cell and a surface protein of the virus.

Methods for producing genetically modified cells or mammals (e.g. transgenic animals) with reduced/increased expression of a given gene are also well known in the art. Some methods for lowering/eliminating levels of chemokine receptors in cells have been described hereinbefore. As for methods for increasing levels of CCRs in cells and/or mammals, these are within the skill of a person working in the art. These may include for instance the use of drugs or compounds such as cell stimulators and activators (e.g. cytokines, chemokines, mediators, proteins, proteoglycans, lectins) and even other viruses, molecular biology techniques (transfection, over expression of genes).

G) Method for Gene Transfer for Genetic Therapy

Since in human and animals the CCR1, CCR2, CCR3, CCR4, CCR5, and CCR8 receptors are expressed on a limited number of specific cells, the invention encompasses the use of a viral particle as a delivery vehicle for introducing an exogenous non-viral nucleic acid molecule in a CCR1-, CCR2-, CCR3-, CCR4-, CCR5-, and/or CCR8-positive cell, the viral particle being capable of entering the cell. The exogenous non-viral nucleic acid molecule may consist of antisense oligonucleotides, double stranded RNAs, and nucleic acid molecules comprising a sequence coding for a therapeutic gene product. For instance, genetically modified HRSV could be used as a vehicle for gene transfer in genetic therapy methods.

The present invention therefore relates to gene therapy and transfection methods as a delivery vehicle for introducing an exogenous non-viral nucleic acid molecule in CCR1-, CCR2-, CCR3-, CCR4-, CCR5-, and/or CCR8-positive cells. The method comprises contacting the cell with a genetically modified virus comprising the exogenous non-viral nucleic acid molecule, the genetically modified virus being capable of infecting the cell. If necessary, the given CCR-positive cells may be obtained by transferring and expressing therein gene(s) of the CCR receptors.

### EXAMPLES:

Although any method and material similar or equivalent to those described herein can be used in the practice or testing of the present invention, the preferred methods and materials are described.

Objectives

Respiratory syncytial virus (RSV) infects all infants by the age of three years old and commonly re-infects children, adults, the immunosuppressed and the elderly. This virus has a propensity to infect epithelial cells. Severe RSV infection in infants is called bronchiolitis and predisposes to the development of asthma. Interestingly, patients with asthma have exacerbations of their disease when they are affected by viral infections of the airways like RSV. Since chemokine receptors have been shown to be important in certain viral infections like HIV, we assessed whether several chemokine receptors were present in the airways of patients with asthma.

B) Reagents and Methods

L-glutamine, penicillin, streptomycin, Hank's balanced salt solution (HBSS), trypsin ethylenediamine tetra-acetic acid (EDTA) were purchased from Sigma chemical Co. Dulbecco's modified minimum essential medium (DMEM), fetal calf serum (FCS), bovine serum albumin (BSA), Superscript II, Taq polymerase, dNTPs, TRIzol reagent were purchased from Life technologies (Gaithersburg, Md.). Rat anti-human CCR3 (clone 61828.111), Mouse anti-human CCR-5 (clone 45502.111), Mouse anti-human CXCR-3 (clone 49801.111), Mouse anti-human CXCR-4 (clone 12G5), recombinant stromal derived factor-1α (SDF-1a), Eotaxin, Macrophage inflammatory protein 1β (MIP-1β) and IP-10 were purchased from R&D systems (Minneapolis, US). Monoclonal antibody to RSV-G protein (Mab858-2) and anti-RSV F-protein monoclonal (Mab858-1) antibodies were from Chemicon International, Temecula, Calif. HRP-conjugated anti-mouse antibodies (Santa Cruz Biotechnology, California, USA). Horse anti-mouse IgG antibody conjugated to biotin (Dako), Streptavidin-alkaline phosphatase (AP) conjugate (Dako), Fast Red substrate for AP (Sigma). A549 type II alveolar epithelial cells and Hep2 epithelial cells were purchased from ATCC. GHOST cells were a gift from the NIH AIDS reagents program.

All cell types used in this work were grown as monolayers in plastic tissue culture flasks at 37° C., 5% CO₂. A549 cells, derived from a lung adenocarcinoma with the alveolar type II cell phenotype (ATCC) were cultured in F12K medium (Life Technologies, Gibco BRL) with 10% heat inactivated FBS, GHOST cells (NIH AIDS reagents) were cultured in DMEM supplemented with 10% heat inactivated fetal calf serum; Hep-2 cells were grown in Eagle's modified essential medium EMEM (Life Technologies, Gibco BRL) supplemented with 10% fetal bovine serum. The culture medias were supplemented by 2 mM L-glutamine, 100 U/ml penicillin and 100 μg/ml streptomycin to maintain the cells and puromycin (with G418 for transfected cells) were added to GHOST cell culture medium. The cells were sub-cultured following brief treatment with trypsin/EDTA.

The A2 strain of RSV was purchased from ATCC. The RSV was inoculated into a sub-confluent Hep-2 cell monolayer. After adsorption for 2 h at 37° C., 10% EMEM was added, and the infection was allowed to proceed for 3 days until the entire monolayer shows cytopathic effects. The contents of the flask was resuspended in 1 ml aliquots, quick-frozen with alcohol/dry ice, and stored at −80° C. The virus titer of RSV was assessed by quantitative plaque forming assay and maintained titers in the range of 10⁶ to 10⁸pfu/ml for more than 6 months at −70° C.

Because RSV-G protein has a physical interaction with respiratory epithelial cells, we have tested its ability to interact with CCR3 receptor, and demonstrated the specificity of this interaction by using a combination of inhibition, immunoprecipitation and western blotting techniques adapted from Steven A. Feldman (Feldman, S. et al. 1999. J. Virol. 73: 6610-17). A549, Hep2, or GHOST cells were infected by RSV A2 strain at different multiplicity of infection (MOI). The results presented in this work were performed at a MOI of 1:0.1. The MOI 1:0.1 is the cut off of permissive and restrictive infection. The cells expressing the chemokine receptor CCR3 (Ghost-CCR3+, Hep2 and A549) were found permissive at a MOI 1:0.1 and the chemokine receptor CCR3 is considered as the high affinity coreceptor for RSV infection. Infection experiments undertaken with GHOST cells expressing only one of the following chemokine receptors the CCR1, CCR2b, CCR4, CCR5, CCR8 or CXCR4, were performed at a MOI of 1:2. These GHOST cells (CCR1, CCR-2b, CCR-4, CCR-5, CCR-8 and CXCR4), were not infected at a MOls of 1:0.1 and 1:1. The chemokine receptors CCR1, CCR2, CCR4, CCR5, CCR8 were considered subsequently as low affinity coreceptors in regard to RSV infection. GHOST parental cells, which do not express any chemokine receptor were always used as control in all these experiments. These cells were not infected by RSV at a MOI 1:5 and lesser (1:4, 1:3, 1:2, 1:1 and 1:0.1).

Cells were infected by RSV for 2 hr incubation in the presence or the absence of 2 μg/ml of Eotaxin, or of PBS solution and cultured for 3 days. Cells were washed three times in cold PBS and lysed in RIPA buffer containing 0.5% Triton X-100, 150 mM NaCl, 200 mM boric acid (pH8), 5 mM EDTA, 5 mM sodium fluoride and 1 mM sodium vanadate at a ratio of 30-50 million cells per 1 mL of lysis buffer. Lysates were spun for 30 min at 14000 RPM in a microfuge to remove cell nuclei prior to immunoprecipitations. Immunoprecipitations were performed by binding the anti-CCR3 antibody to protein A or G-sepharose beads (Pharmacia Biotechnology Inc.), washing the beads twice with lysis buffer and then incubating the beads with the cell lysate for 2-14 h, rocking at 4° C. Beads were then washed five times with lysis buffer, resuspended in 2× Laemmli sample buffer (1M Tris, 25% glycerol, 0.5% SDS, 15% 2-mercaptoethanol, 0.1 mg/ml Bromophenol blue) and boiled for 10 min at 70° C., resolved on 10% SDS-PAGE. SDS-PAGE and Western transfer were performed using standard methods. All Western transfers were to Immobilon PVDF membranes (Millipore). Immunoblotting was performed according to the protocols supplied with the enhanced chemiluminescent detection kit (Boehringer). The blots were blocked in TBS containing 5% BSA (Sigma), 0.05% Tween 20™ for 90 min. at room temperature and the anti-RSV-G protein antibodies were added in the same buffer at 4° C. for 12 hours (1:1000 dilution). HRP-conjugated anti-mouse antibodies (1:1000) was added for another hour and washed 3 times in TBS containing 0.05% Tween 20. The blots were developed by enhanced chemiluminescence (Boehringer) and exposed to X-ray film (Kodak).

C) Results And Discussion

Here we show that the chemokine receptor CCR3 does associate physically with the RSV-G protein and function as a virus coreceptor for cell entry and infectivity.

The RSV which belongs to Pneumovirus genus of the Paramyxovirus family, is the major cause of acute lower respiratory tract illness in infants and young children. The RSV envelope contains two glycoproteins, G and F that are responsible, respectively, for virus attachment to the cell and for cell fusion. A third protein, named SH, has an unknown function. The RSV-G protein has unusual features compared to other Paramyxovirus glycoproteins. The RSV-G protein is synthesized as a precursor of 30 kDa that is modified by the addition of N-linked sugars to form an intermediate of 45 kDa. These sugars convert to the complex type, and O-linked sugars are added to yield a mature molecule of approximately 90 kDa. The RSV-G protein appears to be linked to the pathogenesis of RSV disease, it has been linked to the induction of Th2 cytokines and development of eosinophilic pulmonary infiltrates in mice. The RSV-G protein has been also associated with altered tumor necrosis factor (TNF) and interferon-gamma (IFN-g) expression; altered pulmonary polymorphic neutrophils (PMNs) and natural killer (NK) cell trafficking, altered chemokine mRNA expression of macrophage inflammatory protein 1-alpha and MIP-2, monocyte chemoattractant protein 1 (MCP-1) and IFN-inducible protein 10 (IP-10) by bronchoalveolar leukocytes (Tripp et al., Nature Immunol., 2001, 2, 732-738). The above imune responses are suggestive of chemokine function and structural features of the RSV-G protein have marked similarities to chemokines. Of interest is like all chemokines, RSV-G has a heparin binding domain (HBD), (amino acid positions 182-186) that interact with glycosaminoglycans (GAGs) on cells (Feldman et al., 1999, G. J. Virol., 73, 6610-6617). Given the marked consensus between the RSV-G and chemokines for GAGs requirement for function, we examined the possible use of chemokine receptors as co-receptor for RSV infection.

Hep2 cells are known for being a permissive target cell for RSV infection and these cells express several chemokine receptors. Serial rounds of infections were performed with Hep-2 cells at different MOIs, ranging from 1:5 to 1:0.1. Hep-2 cells were systematically infected at these ranges of MOI. Subsequent experimental infection of Hep-2 cells with RSV were realized at a MOI 1:0.1. We investigated the role of the chemokine receptor CCR3 in supporting RSV infection. Immunoprecipitation technique followed by analysis of the immunoprecipitated complexes by SDS-PAGE were performed in order to gain insight into the physical contact between the RSV-G protein and the CCR3 receptor. Immunoprecipitation of the immune complexes will allow to assess, using SDS-PAGE and a specific antibody for the RSV-G protein, whether or not the contact of the virus glycoprotein with the cell receptor occurred. The results shown in FIG. 1 indicated that RSV-G mAb detects the RSV-G protein among the anti-CCR3 mAb immunoprecipitated protein complexes (FIG. 1 lane 4). These results are a first line of evidence toward a physical contact between the CCR3 receptor and the RSV-G protein in Hep-2 cells. Indeed, this interaction was found to be specific for CCR3 receptor since the RSV-G mAb failed to recognize the RSV-G protein in the extracts from Hep2 cells infected by RSV in the presence of 2 μg/ml of Eotaxin (FIG. 1 lane 3). Suggesting that Eotaxin, a CCR3 specific ligand, was able to inhibit the RSV infection following its binding to its specific receptor which is probably used by RSV as a coreceptor for host cell entry. We noticed here that although the monoclonal antibody to RSV-G protein was diluted to 1:5000, it still cross reacted with a determinant of 43-45 kDa in all non infected or control cell types used in this work (see FIG. 1 lane 2). However, keeping in mind that all cell extracts were normalized to the same quantity of proteins before being loaded into SDS-PAGE. Therefore, the high intensity of the band, recognized by anti-RSV-G, and corresponding to the RSV-G protein precursor allowed us to differentiate between real infection and the background due to cross reactivity. We assessed if the CCR3 receptor was involved in virus infectivity of epithelial cells by performing RSV infectivity inhibition tests as described above. RSV A2 were plated onto replicate Hep-2 cell cultures in the presence of different concentrations of Eotaxin ranging from 0.01 to 1.0 μg/ml, or of the control PBS solution (FIG. 2). The cultures were washed 3 times with pre-warmed PBS and overlaid with RPMI nutrient media containing 10% fetal calf serum and 0.75% methylcellulose to prevent virus movement by diffusion. Three days post infection, the cultures were fixed in 4% paraformaldehyde and stained with 1% crystal violet. The plaques (RSV final cytopathic effect, illustrated by wholes within the cells carpet indicating that the virus infection was successful and resulted by viral particles shedding from locally dead cells) were counted by eye and the percent inhibition of virus plaques formation, of Eotaxin-treated compared to PBS-treated cell cultures was determined. Concentrations of Eotaxin greater or equal to 0.5 μg/ml inhibited RSV plaque formation by 80% (FIG. 2) and there were statistically fewer RSV plaques as compared with PBS controls at each concentration of Eotaxin tested (p<0.05, using Student's t-test). This result confirmed the RSV-G protein specific requirement for CCR3 to select and attach to specific target cells and subsequently the achievement of virus cell entry processes. Moreover, our discovery suggested that Eotaxin is a specific competitor for RSV-G binding to CCR3 since this chemokine, known to be a natural ligand for CCR3, was found to inhibit the attachment, entry and cytopathological effects of RSV infection.

We have also assessed whether CCR3 transfected GHOST cells support RSV infection and produce infectious virus. We performed this by using the same methods as described in FIG. 3. The presence of RSV protein was demonstrated 72 hours after infection by immunostaining. The method of immunostaining was as described by Lamkhioued, et al. (J. Immuno. 1997, 159(9):4593-4601); cells were incubated sequentially with (a) anti-RSV F-protein monoclonal antibody (1:1000; first antibody), (b) horse anti-mouse IgG antibody conjugated to biotin (1:5000; second antibody), (c) streptavidin-alkaline phosphatase (AP) conjugate (detector molecule), and (d) Fast Red substrate for AP. A positive reaction for RSV is indicated by the presence of red product and numerous red dots are visible in FIG. 3A. Panel A shows that GHOST cells transfected with the CCR3 receptor are infected by RSV, whereas parental cells that do not contain the CCR3 receptor are not infected (panel C). Panel B shows that the addition of Eotaxin to GHOST/CCR3 cells inhibits infection by RSV, (Panel D: controls).

Similarly, FIG. 4, Panel B shows uninfected Hep-2 cells (these are also CCR3-positive) while Panel A shows RSV-infected Hep-2 cells (same conditions as above). After fixing the cells in 4% paraformaldehyde in phosphate buffered saline (PBS), the presence of virus was detected by immunostaining. When the same experiment was repeated with CCR3+GHOST cells incubated with anti-CCR3 neutralizing antibodies (2 μg, Panel D) or with the control PBS solution, prior to being infected with RSV (Panel C), the antibodies inhibited viral infection by approximately 80% as evidenced by many fewer red dots visible in Panel D vs. Panel C.

The same experiments with anti-CCR3 neutralizing antibodies were repeated in A549 cells (FIG. 5). FIGS. 5A (non infected) and 5B (infected) illustrate that A549 epithelial cells support RSV infection and produce infectious virus as demonstrated 72 hours after infection by immunostaining. FIG. 5C illustrate the neutralizing effect of antibodies directed against the eotaxin receptor (2 μg) on RSV infection. The infection of A549 cells by RSV was blocked by neutralizing antibodies that are specific for CCR3 (5C). However, the infection of A549 cells by RSV was not blocked by neutralizing antibodies that are specific for CCR5 (2 μg, 5D).

Screening of Other Chemokine Receptors

Careful analysis of the RSV-G protein shows that the virus glycoprotein contains a truncated chemokine motif homologous to the N-terminal region of the human chemokine fraktalkine, chemokine domain (FCD). FIG. 6A shows the sequence distribution of charged and hydrophilic amino-acids in the RSV-G and the human chemokine fraktalkine, the natural ligand of the chemokine receptor CX3CR1. Human fraktalkine domain sequence (humFKN), (Harrison et al., J. Biol. Chem., 2001, 276, 21632-21641), is aligned with RSV-G A2 protein segment (A2RSV-G) containing the heparin binding domain (Feldman et al., J. Virol, 1999, 73, 6610-6617). Polar residues are in upper case letters and non polar residues are lower case letters. The fraktalkine chemokine motif (FCM) is in italic, note that Potential N-glycosylation site (NGS; consensus sequence: NxTor S) within the humFKN sequence (NIT) is overlapping with the FCM, are in bold and underlined. The heparin binding motifs (HBM), (consensus sequence: XBBXBX where X is each of amino-acids and B is preferably a basic amino-acid) are in thin letters and underlined. Basic residues (K7, K14, K36, R37, R47, K54 and R74) conserved within murine, rat and human fraktalkine chemokine are starred. Alignment of RSV-G chemokine domain with the human fraktalkine domain shows nearly 42% of sequence homology (FIG. 6A). Structural features shared between the virus glycoprotein and the chemokine Fraktalkine are highlited in FIG. 6A. Of particular interest is the CX3C signature sequence, heparin binding domains, N-glycosylation sites. Interestingly, it was recently shown that the RSV-G protein could contact the chemokine receptor CX3CR1, suggesting that this interaction probably plays a role in the biology of RSV infection (Tripp et al., 2001, Nature Immunol., 2, 732-738). However, RSV is known to infect respiratory tract cells and CX3CR1 is expressed in T cells and endothelial cells of the lung, whether it is expressed on epithelial cells remains to be determined. Our finding that CCR3 support RSV infection is in keeping with the fact that CCR3 is expressed in bronchial epithelial cells (Stellato et al., J. Immunol. 2001, 166, 1457-1461).

All chemokine receptors identified thus far are membrane-bound proteins composed of seven-transmembrane domains and coupled to G-proteins. The major hallmarks of these receptors are as follows. They are nearly 350 amino-acids in length and require the introduction of few gaps in the primary sequence to be aligned to other chemokine receptors. The short extracellular N-terminal region of these receptors is highly acidic and may be sulfated on tyrosine residues (sequence consensus: NXT/S, where N=Asparagine, X=Each of amino-acids, T=Threonine or S=serine) and contains N-linked glycosylation sites (consensus sequence: AYD where A=hydropathic amino acid: Histidine, Arginine, Lysine, Aspartic acid, glutamic acid, Asparagine, Glutamine, Threonine, Tyrosine or Serine; D=Aspartic acid). The C-terminal region of these chemokine receptors contains serine and threonine residues that act as phosphorylation sites for receptor regulation. The remaining part of these receptors is composed of seven alpha-helical transmembrane domains, with 3 intracellular and 3 extracellular (EC) connecting loops composed of hydrophilic (E, D, R, K, H, N, Q, T, S) amino acids, a disulfide bond links highly conserved cysteines in EC1 and EC2; G-proteins are coupled through the C-terminus segment and possibly through the third intracellular loop (Murdoch and Finn for review, Blood, 2000, 95, 3032-3043).

We performed a protein sequence alignment survey between the CCR3 and different chemokine receptors (CCR1, CCR2, CCR4, CCR5, CCR6, CCR7, CCR8; CX3CR1 and CXCR4), in order to delineate segments of these receptors sharing sequence homology to the CCR3. We analyzed the N-terminal regions of these receptors, because it was shown that the N-terminal part of the chemokine receptors CCR5, CCR2, CCR1 and CX3CR1 act as co-receptor for HIV-1 infection and for specific chemokine binding. Moreover, swapping and mutagenesis analysis of the chemokine fractalkine showed that positively charged amino-acids, localised at the fractalkine chemokine domain are involved in the CX3XR1 interaction (Mizoue et al., Biochem., 1999, 38, 1402-1414; Dragic, J. G. Virol., 2001, 82, 1807-1814), (FIG. 6A).

FIG. 6B shows the alignment of the chemokine receptor N-terminal regions starting at the first Methionine of each receptor. Polar amino acids present in the N-terminal segment of the receptor CCR3 are highlighted by (1) whereas potential N-glycosylation sites (NxT or S) are underlined in italic. Putative heparin binding domain are underlined in thin letters and the amino acids subdomains defining the groups of chemokine receptors are noted as I, II, III, IV and V. The CCR3 sulfatation tyrosine residue (Y17) is starred. The alignment shown in FIG. 6B takes into account the distribution of polar amino acid residues (E, D, T, S, Q, N), since such residues are hydrophilic and able to form stable interactions with positively charged amino acids (K, R, H). The alignment shows a conserved structural homology among the receptors CCR3, CX3CR1, CCR5, CCR8 (hereafter mentioned as the group A) and to a lesser extent CCR1 (group B), CCR4 and CCR2b (group C). Indeed, the amino acids composition and distribution, namely polar residues are well conserved among these group of receptors. The sequence of CCR1 harbors an N-glycosylation site which was not found in CCR3, CX3CR1, CCR5 and CCR8, and subsequently diminished the score of homology towards the group A receptors. The receptors CCR4 and CCR2b have a long stretch of amino acids (block IV), and CCR4 has an N-glycosylation site while CCR2b contains a HBD, which make them divergent from groups A and B. The receptors CCR6, CCR7 and CXCR4 were found completely divergent from the rest of receptors (group D) because the distribution pattern of polar amino acids is different in blocks I, II, III and IV.

We subsequently analyzed the extracellular domains of these receptors, because these domains are involved in protein-protein interactions. The FIG. 6C shows the alignment of the first extracellular (EC1) domains of the chemokine receptors. Polar amino acids are in upper case letters. Heparin binding sites (XBBXBX) are underlined whereas the CCR6 putatif N-glycosylation site (NXT or S) is undelined and in italic. As it can be seen, the CCR3-EC1 and the CX3CR1-EC1 are rich in basic amino acids and contain a HBD which again place them as highly conserved amino acid composition receptors. The CCR1-EC1 sequence contains a putative HBD, but the abundance of basic amino acids render it less closer to CCR3 and CX3CR1. CCR5, CCR4, CCR8 and CCR2b form the intermediate group, in which amino acid sequences showed few basic amino acids. Finally, the CCR6, CCR7 and CXCR4 seem to be divergent, they contain N-glycosylation sites (CCR6 and CCR7) and contain few polar amino acids.

FIG. 6D shows the sequence alignment of the chemokine receptors EC2 domain. Heparin binding motifs are underlined in thin letters. N-glycosylation sites are in italic and underlined. It is interesting to note that the heparin binding motif in CCR4-EC2 overlaps with N-glycosylation site. As it can be further appreciated, the CCR3-EC2 is highly rich in acidic amino acids and does not contain HBD neither N-glycosylation sites which place it as a unique domain. The structure and amino acid composition of CX3CR1 and CCR5 is quite similar, taking into account the localization and juxtaposition of HBD and the amount of acidic residues. On the other hand, CCR1, CCR6 and CCR8 contain HBDs at the C-terminal part of the EC2. CCR4 is placed in an intermediate position, it contains a HBD at the N-terminal part of EC2, as CCR5 and CX3CR, and an N-glycosylation site at the C-terminal region of EC2. Finally CCR2b, CCR7 and CXCR4 form a divergent group which do not fit with any mentioned groups in the EC2 region.

FIG. 6E shows the alignment of chemokine receptors EC3. Polar amino acids are in upper case letters and the putative N-glycosylation sites are in italic and underlined. To better delineate sequence homology in this extracellular region we considered just the C-terminal part of EC3, because the N-terminal region of this segment is highly divergent. The CX3CR1, CCR3, CCR1, CCR7, CCR4 and CCR6 have a EC2-C-terminal region rich in basic and acidic residues, which place these receptors in the same group of homology. Finally, CXCR4, CCR2b, CCR5 and CCR8 contain few if any charged amino acids which place them in a second group of homology.

The overall analysis indicates that several structural features are shared between CCR3, CCR1, CCR2b, CCR4, CCR5, CCR8 and CX3CR1, suggesting conserved function between these receptors. This is not surprising since it is well known that some of these receptors share the specific binding for shared chemokines (see the aforementioned chemokine receptor ligands).

FIG. 7 shows a Western blot performed to assess if the cells expressing the chemokine receptors CCR4 or CCR5 were able to support RSV infection. We performed GHOST cells infection at MOls 1:2, 1:1, 1:0.1, from this only MOI of 1:2 were relevant and ended in cell infection. The Western blot in FIG. 8 indicated that the cells were infected since we could detect the RSV-G protein using antibodies to RSV-G. The same experiments were performed with GHOST cells expressing CCR1 or CCR2b (FIG. 8), here also infections at a MOI of 1:2 were relevant and we could detect by Western blot the presence of RSV-G protein in the cell extracts.

D) Conclusions

These experimental results identify the cellular CCR3 receptor as a viral coreceptor for RSV. Anti-CCR3 antibodies specifically inhibited infection with RSV, by binding to the CCR3 receptor and preventing the subsequent binding of RSV to this receptor, most likely through steric hindrance. In addition, we have found by analysis of sequence homology that there are other potential coreceptors for RSV: CCR1, CCR2, CCR4, CCR5 and CCR8. These results were confirmed by visual inspection of the infection of GHOST cells bearing these receptors and by showing that RSV protein was in fact found in the GHOST CCR1, CCR2, CCR4 and CCR5 transfected cells when assessed by trnasfer and Western analysis.

The interaction between chemokine receptors and viruses may be part of a more general strategy that is employed by other microorganisms and uses the plasticity of genomes to elude the immune system of their hosts. In addition, as shown here, a single virus may employ different receptors to infect different cell types or host species. By binding through chemokine receptors, RSV has the potential to up-regulate the inflammatory reaction by inducing the chemotaxis of inflammatory cells. The public health implications (prevention of viral infection by blocking binding and/or fusion of RSV to airway epithelium) are considerable. In addition, since RSV seems to infect mostly the respiratory epithelium, it will be possible to design aerosolized drugs to prevent or treat RSV infection by preventing the attachment of RSV to it's receptor(s).

While several embodiments of the invention have been described, it will be understood that the present invention is capable of further modifications, and this application is intended to cover any variations, uses, or adaptations of the invention, following in general the principles of the invention and including such departures from the present disclosure as to come within knowledge or customary practice in the art to which the invention pertains, and as may be applied to the essential features hereinbefore set forth and falling within the scope of the invention or the limits of the appended claims.

1-85. (canceled)

86. A method for modulating viral infection of a cell, comprising modulating a binding interaction between a cell chemokine-receptor and a surface protein of said virus, said cell chemokines-receptor comprising an amino acid sequence having at least 38% identity with SEQ ID NO:6, with the proviso that said virus is not HIV.

87. The method of claim 86, wherein said cell chemokine-receptor is selected from the group consisting of CCR1, CCR2, CCR3, CCR4, CCR5, and CCR8.

88. The method of claim 87, wherein said cell chemokine-receptor consists of CCR3.

89. A method of modulating a pneumovirus infection of a cell, comprising modulating a binding interaction between at least one chemokine-receptor of said cell and a surface protein of said pneumovirus, wherein said cell chemokine-receptor is selected from the group consisting of CCR1, CCR2, CCR3, CCR4, CCR5, and CCR8.

90. The method of claim 89, comprising the step of contacting said cell with a CCR-ligand that recognizes at least one of said cell chemokine-receptor, said contacting step being carried out under conditions sufficient for said CCR-ligand to bind at least one of said cell chemokine-receptor, whereby the ability of said pneumovirus to subsequently bind said cell is attenuated.

91. The method of claim 90, wherein said CCR-ligand is selected form the group consisting of: eotaxin I, eotaxin 2, eotaxin 3, monocyte chemoattractant protein-1 (MCP-1), MCP-2, MCP-3, MCP-4, MCP-5, MIP-1α, MIP-1β, I-309, regulated on activation normal T-cell expressed and secreted (RANTES), TARC, antibodies and fragments thereof which specifically recognize said cell receptor(s), and polypeptides having a binding activity to an extracellular domain of said receptor(s).

92. The method of claim 91, comprising the step of reducing intracellular levels of at least one of said cell chemokine-receptor with antisense molecules introduced or expressed into said cell, thereby limiting pneumovirus ability to infect said cell.

93. The method of claim 89, comprising the step of contacting said pneumovirus with a virus-ligand that recognizes a surface protein of said pneumovirus under conditions sufficient for said virus-ligand to bind said pneumovirus surface protein and subsequently attenuates binding of said pneumovirus to said cell chemokine-receptor(s).

94. The method of claim 93, wherein said virus-ligand is selected from the group consisting of:
a. an antibody or fragment thereof which specifically recognizes said virus surface protein; and
b. a CCR1, CCR2, CCR3, CCR4, CCR5 and/or CCR8 receptor or a functional fragment thereof.

95. The method of claim 94, wherein said antibody or fragment thereof specifically binds to a RSV-G or RSV-F protein.

96. The method of claim 95, wherein said virus consists of the Human Respiratory Syncytial Virus (HRSV).

97. A method of reducing the initiation or spread of a respiratory tract disease due to human RSV, comprising administering to a human an antiviral agent comprising a virus-ligand which exhibits the ability to bind to HRSV and reduce infectivity thereof, said virus-ligand exhibiting the ability to bind to a HRSV surface protein involved in binding of the HRSV to a cell chemokine-receptor selected from the group consisting of CCR1, CCR2, CCR3, CCR4, CCR5, and CCR8.

98. A method for detecting the presence of a pneumovirus in a biological sample, comprising the steps of:
a. contacting the biological sample with a cell expressing at least one cell chemokine-receptor selected from the group consisting of CCR1, CCR2, CCR3, CCR4, CCR5, and CCR8, said contacting step being carried out under conditions sufficient for pneumovirus(es) contained in said sample to infect said cell; and
b. detecting the presence of pneumovirus(es) in said cell.

99. The method of claim 98, wherein said cell is selected from the group consisting of: Hep-2 cells, A549 cells, and GHOST cells transfected with the CCR1, CCR2, CCR3, CCR4, CCR5 and/or CCR8 receptor(s).

100. The method of claim 99, wherein said cell has been pretreated with agent(s) capable of increasing or permitting the expression of said chemokine-receptor(s) on the cell surface.

101. A method for selecting an antiviral compound that is capable of reducing viral infection of a cell by a pneumovirus, the cell expressing at least one cell chemokine-receptor selected from the group consisting of CCR1, CCR2, CCR3, CCR4, CCR5 and CCR8, the method comprising:
a. contacting a functional virus-ligand and a surface protein of a virus in the presence of a potential antiviral compound, said surface protein being involved in binding of the virus to at least one of the chemokine-receptor expressed by the cell;
b. measuring a binding interaction between said virus-ligand and said viral surface protein; whereby an antiviral compound is selected when said binding interaction is measurably reduced in presence of said potential antiviral compound.

102. The method of claim 101, wherein said virus-ligand is selected from the group consisting of:
a. an antibody or a fragment thereof which specifically recognizes said pneumovirus surface protein; and
b. a CCR1, CCR2, CCR3, CCR4, CCR5 and/or CCR8 receptor or a functional fragment thereof.

103. The method of claim 102, wherein said pneumovirus consists of the Human Respiratory Syncytial Virus (HRSV).

104. A composition for treating and/or preventing infection by a pneumovirus, said composition comprising a CCR-ligand and a pharmaceutically acceptable carrier, said CCR-ligand exhibiting the ability to bind to at least one chemokine-receptor selected from the group consisting of CCR1, CCR2, CCR, CCR4, CCR5, and CCR8, and thereby reduce infectivity of the pneumovirus.

105. The composition of claim 104, wherein said composition consists of a spray for application to airway tissues susceptible to infection by HRSV.

106. A method of attenuating the ability of a pneumovirus to infect a mammalian cell expressing at least one chemokine-receptor selected from the group consisting of CCR1, CCR2, CCR3, CCR4, CCR5, and CCR8, the method comprising exposing said cell to a CCR-ligand that recognizes at least one of said cell chemokine-receptor, said exposition being carried out under conditions sufficient for said CCR-ligand to bind said at least one of said cell chemokine-receptor, whereby the ability of said pneumovirus to subsequently infect said cell is attenuated.

107. The method of claim 106, wherein said CCR-ligand is selected from the group consisting of: eotaxin 1, eotaxin 2, eotaxin 3, monocyte chemoattractant protein-1 (MCP-1), MCP-2, MCP-3, MCP-4, MCP-5, MIP-1α, MIP-1β, I-309, regulated on activation normal T-cell expressed and secreted (RANTES), TARC, antibodies and fragments thereof which specifically recognize said cell receptor(s), polypeptides having a binding activity to an extracellular domain of said receptor(s).

108. A method of attenuating the ability of a pneumovirus to bind a mammalian cell, comprising a step selected from the group consisting of:
a. exposing said cell to a CCR-ligand that recognizes at least one cell chemokine-receptor selected from the group consisting of CCR1, CCR2, CCR3, CCR4, CCR5, and CCR8, said exposition being carried out under conditions sufficient for said CCR-ligand to bind said cell receptor;
b. exposing said virus to a virus-ligand that recognizes a surface protein of the pneumovirus, said exposition being carried out under conditions sufficient for said virus-ligand to bind said pneumovirus surface protein and subsequently attenuates binding of pneumovirus to at least one cell chemokine-receptor selected from the group consisting of CCR1, CCR2, CCR3, CCR4, CCR5, and CCR8; and
c. reducing intracellular levels of at least one cell chemokine-receptor selected from the group consisting of CCR1, CCR2, CCR3, CCR4, CCR5, and CCR8;
thereby limiting the pneumovirus ability to bind said mammalian cell.


SEQUENCE LISTING (20 sequences)

SEQ ID NO: 1 - DNA, 2156 bp, Homo sapiens
  CDS (63)..(1130)
ggcacgagcccagaaacaaagacttcacggacaaagtcccttggaaccagagagaagccgggatggaaactccaaacaccacagaggactatgacacgaccacagagtttgactatggggatgcaactccgtgccagaaggtgaacgagagggcctttggggcccaactgctgccccctctgtactccttggtatttgtcattggcctggttggaaacatcctggtggtcctggtccttgtgcaatacaagaggctaaaaaacatgaccagcatctacctcctgaacctggccatttctgacctgctcttcctgttcacgcttcccttctggatcgactacaagttgaaggatgactgggtttttggtgatgccatgtgtaagatcctctctgggttttattacacaggcttgtacagcgagatctttttcatcatcctgctgacgattgacaggtacctggccatcgtccacgccgtgtttgccttgcgggcacggaccgtcacttttggtgtcatcaccagcatcatcatttgggccctggccatcttggcttccatgccaggcttatacttttccaagacccaatgggaattcactcaccacacctgcagccttcactttcctcacgaaagcctacgagagtggaagctgtttcaggctctgaaactgaacctctttgggctggtattgcctttgttggtcatgatcatctgctacacagggattataaagattctgctaagacgaccaaatgagaagaaatccaaagctgtccgtttgatttttgtcatcatgatcatcttttttctcttttggaccccctacaatttgactatacttatttctgttttccaagacttcctgttcacccatgagtgtgagcagagcagacatttggacctggctgtgcaagtgacggaggtgatcgcctacacgcactgctgtgtcaacccagtgatctacgccttcgttggtgagaggttccggaagtacctgcggcagttgttccacaggcgtgtggctgtgcacctggttaaatggctccccttcctctccgtggacaggctggagagggtcagctccacatctccctccacaggggagcatgaactctctgctgggttctgactcagaccataggaggccaacccaaaataagcaggcgtgacctgccaggcacactgagccagcagcctggctctcccagccaggttctgactcttggcacagcatggagtcacagccacttgggatagagagggaatgtaatggtggcctggggcttctgaggcttctggggcttcagtcttttccatgaacttctcccctggtagaaagaagatgaatgagcaaaaccaaatattccagagactgggactaagtgtaccagagaagggcttggactcaagcaagatttcagatttgtgaccattagcatttgtcaacaaagtcacccacttcccactattgcttgcacaaaccaattaaacccagtagtggtgactgtgggctccattcaaagtgagctcctaagccatgggagacactgatgtatgaggaatttctgttcttccatcacctccccccccccgccaccctcccactgccaagaacttggaaatagtgatttccacagtgactccactctgagtcccagagccaatcagtagccagcatctgcctccccttcactcccaccgcaggatttgggctcttggaatcctggggaacatagaactcatgacggaagagttgagacctaacgagaaatagaaatgggggaactactgctggcagtggaactaagaaagcccttaggaagaatttttatatccactaaaatcaaacaattcagggagtgggctaagcacgggccatatgaataacatggtgtgcttcttaaaatagccataaaggggagggactcatcatttccatttacccttcttttctgactatttttcagaatctctcttcttttcaagttgggtgatatgttggtagattctaatggctttattgcagcgattaataacaggcaaaaggaagcagggttggtttcccttctttttgttcttcatctaagccttctggttttatgggtcagagttccgactgccatcttggacttgtcagcaaaaaaaaaaaaaaaaa

SEQ ID NO: 2 - protein, 355 aa, Homo sapiens
METPNTTEDYDTTTEFDYGDATPCQKVNERAFGAQLLPPLYSLVFVIGLVGNILVVLVLVQYKRLKNMTSIYLLNLAISDLLFLFTLPFWIDYKLKDDWVFGDAMCKILSGFYYTGLYSEIFFIILLTIDRYLAIVHAVFALRARTVTFGVITSIIIWALAILASMPGLYFSKTQWEFTHHTCSLHFPHESLREWKLFQALKLNLFGLVLPLLVMIICYTGIIKILLRRPNEKKSKAVRLIFVIMIIFFLFWTPYNLTILISVFQDFLFTHECEQSRHLDLAVQVTEVIAYTHCCVNPVIYAFVGERFRKYLRQLFHRRVAVHLVKWLPFLSVDRLERVSSTSPSTGEHELSAGF

SEQ ID NO: 3 - DNA, 1979 bp, Homo sapiens
  CDS (81)..(1163)
caggactgcctgagacaagccacaagctgaacagagaaagtggattgaacaaggacgcatttccccagtacatccacaacatgctgtccacatctcgttctcggtttatcagaaataccaacgagagcggtgaagaagtcaccaccttttttgattatgattacggtgctccctgtcataaatttgacgtgaagcaaattggggcccaactcctgcctccgctctactcgctggtgttcatctttggttttgtgggcaacatgctggtcgtcctcatcttaataaactgcaaaaagctgaagtgcttgactgacatttacctgctcaacctggccatctctgatctgctttttcttattactctcccattgtgggctcactctgctgcaaatgagtgggtctttgggaatgcaatgtgcaaattattcacagggctgtatcacatcggttattttggcggaatcttcttcatcatcctcctgacaatcgatagatacctggctattgtccatgctgtgtttgctttaaaagccaggacggtcacctttggggtggtgacaagtgtgatcacctggttggtggctgtgtttgcttctgtcccaggaatcatctttactaaatgccagaaagaagattctgtttatgtctgtggcccttattttccacgaggatggaataatttccacacaataatgaggaacattttggggctggtcctgccgctgctcatcatggtcatctgctactcgggaatcctgaaaaccctgcttcggtgtcgaaacgagaagaagaggcatagggcagtgagagtcatcttcaccatcatgattgtttactttctcttctggactccctataacattgtcattctcctgaacaccttccaggaattcttcggcctgagtaactgtgaaagcaccagtcaactggaccaagccacgcaggtgacagagactcttgggatgactcactgctgcatcaatcccatcatctatgccttcgttggggagaagttcagaaggtatctctcggtgttcttccgaaagcacatcaccaagcgcttctgcaaacaatgtccagttttctacagggagacagtggatggagtgacttcaacaaacacgccttccactggggagcaggaagtctcggctggtttataaaacgaggagcagtttgattgttgtttataaagggagataacaatctgtatataacaacaaacttcaagggtttgttgaacaatagaaacctgtaaagcaggtgcccaggaacctcagggctgtgtgtactaatacagactatgtcacccaatgcatatccaacatgtgctcagggaataatccagaaaaactgtgggtagagactttgactctccagaaagctcatctcagctcctgaaaaatgcctcattaccttgtgctaatcctctttttctagtcttcataatttcttcactcaatctctgattctgtcaatgtcttgaaatcaagggccagctggaggtgaagaagagaatgtgacaggcacagatgaatgggagtgagggatagtggggtcagggctgagaggagaaggagggagacatgagcatggctgagcctggacaaagacaaaggtgagcaaagggctcacgcattcagccaggagatgatactggtccttagccccatctgccacgtgtatttaaccttgaagggttcaccaggtcagggagagtttgggaactgcaataacctgggagttttggtggagtccgatgattctcttttgcataagtgcatgacatatttttgctttattacagtttatctatggcacccatgcaccttacatttgaaatctatgaaatatcatgctccattgttcagatgcttcttaggccacatccccctgtctaaaaattcagaaaatttttgtttataaaagatgcattatctatgatatgctaatatatgtatatgcaatataaaatttag

SEQ ID NO: 4 - protein, 360 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRRYLSVFFRKHITKRFCKQCPVFYRETVDGVTSTNTPSTGEQEVSAGL

SEQ ID NO: 5 - DNA, 5791 bp, Homo sapiens
  CDS (4015)..(5082)
ttcttcctaaatttatttacaaatgtaacacaattccacccaaacttatgtttttataagtaattgagtagatgatcctaaagtttaataaaacaaatggctctaataggtaagacatttggaaatgtataatgaaagggagttgcataataagatcatctatataaatcatctaataaatctacaataaaaagtgtctctagcacagaaataagatatcaatagaatataaggtacaaaatcagattcaggaacattaaagaatatacgacaaaggtgatatttcaagcccaaaggggagaagatggttattcaacacatagtgttttaaaatttgtcagataagaatggagaggaggaggctcctctcctctgaccccagggaatgtgagaagagacacagtggttatgaaaggaagcagtcacacctgtggatccctaccttccccatcagagctagggggcatggagcgctctctgctaagatggggacccccaaggaatgtctccctgtggggcacttccttaccagatgggatggccagtgcggttaagttggtggtcaggcagaaaaaaaagatctagtttgtactcttgagagttcctcggtttgttcatggcatgggcagggagtcaaggagcagcagccttgcctcagtgcctaccagtgcaggaaaaggtgcatagcctgggccagggccagggccctggtggaggcgtagtggtaacagagagggctctccattccagcccaaggaagactaagaatgaatacctcatgagtatattagctacaaaccaccacagcaggttccagaaaaaggctcagcgttggaaccaggtcacccccactcagcagacaccagtcatataaatcaaggaccaacaggagacaggaacacccccttcccactctgccccatgtctcaagttgtagtggcccttcctccagatctctgccaccatcttagaaaggaacactgaaagaagaaactgaaattataagctgacagcataaagaggatgagtaaaacctaaaatcattgttcaaatgaatgaatcaagagaagtttaaaccactttggactaaaatgtgtgaatcctttttcctgctatccagcagatgagaagctggtaacagagaccaaaatagtttggagactaaagaatcattgcacatttcactgctgagttgtattgtgagtaattttagttgacctcacttttgtaaatcttgcacacgggcatccatatctgcacagagatatgttaacagtggtaaatgctgcatgaggagattgggtgatttttactttcgtttttgtgctcttctttcttattgttcttacttatttacgattaccctatcgttttccaaaatgtaaaaggccattttgaaagcctaattcaaacctcttcactattttgtatctaagtattcaccttgattgagactgggtagacaggtgaaaaccatatcaggtttttaattttttaatttttaattatttatttatttatttattttttgagatggagtctggctgtcgcccaggctggagtgcagcggcgtgatcacagttcactgcagcctcaaccttctaggctcaagggattctcccacctcagccccccaagtagttgggaccacacgtatgcgccaccatgcctggctaatttcttatttttttgtagagataggatctcactatattgtccaggctggtcttgaattcctgggctcaggtgagcctcccacctgggcctcccaaagtactgggattacaggcatgagccaaggtcccctgcccatatgagattttctgtctctgatcccatgcagctagtaatcaaggacttggctgctgactctggaggacctgcatgctttcttgagctgtgaacttcagtgctaaaagctcataggcagccctgaaacccaaaccaaaaggttctatggtttatcatcccgatcatgttgattttatagaaataacacatgaattaaagacactaccctcaaactgagcaaaacttaagtaatttttttaaagtttgacctgtttttaaatcactcttggagaaaaaggaaaataaatacaaataattaacggtgaatacaggctactatacctttgttctccagaattagcagttctgttcttttcttgctttagatgctgaagtgcagaaggacactctgtgattgtacgtgtgtaactgacaaaatgtgtattttttttctcagctgctatggattggattatgctattatgaataagaatgctgatgggagcacacacaaaccatttgttcctcagtccattttcctcctcaaaagcctggaatgtgccattgatcagtgggagatgtacctggacagacccatgaaaagagatcaacaagttccacccaagggaccctatttttcctaatttcatttgaaatggcttctaattgtccttctttcattcctgcttcctaccagttttacagctttttctggtttcaaatgtgaactcacatacactctcatttttcctcatcacaaccccaagtgacccaatggtcctcactttcgatataagtaaaggaggctctgcattaagggcttgtccaaggcacgcagctgagaggcgctaggactggctccatttccatctctattctcactgactttgactacccagaaccccaacatgtggggcctcagtattcgatcaattattctattaagaagcaaaaacaattccccgcattggccccagttattaagcatttctcagatttaccttgagaaatgcccatcggcctgtatattcacatcttcacccttgtcccttcctcctagaaaggagaaagtcagttggatgccctctgaggaactagtgcatggcttaactgtccttccatgactcctgccttatctgttttctattttcctccttttccaccgaagtctataatctcaagaaaagcaggcactggccttagggctcctggcctaagaaatatcaagtccagtgagaaatcccattgactgacccctcctgcttacccctttgtgatggagaagctcccaggggtttgctttttgcatgttaccaggcctaactcagcatcaccaggggcaagaaaaggaaagtaacctaaactaatgctgcttataattgtaattattgtaatagttaattactgtgattgtacatgtgtaacagacaaaatgtgtatttttttcacagctgctgtggattggattatgccatttggaataagaatgctgttaagagcacacaagccaggttcctcaagtccgtagcaaatttttcaaaagttaaatttaaaaatcactacatttgaatctagtgacaggagaaatggacatggatagagactaaagatctagcccaaattttatatttacttgttagaggattttgaacaaattactaaatttcttcaaggttcaatttccccattaactataatgaatggctcatcattatggggccctggagaagcataattacttgtaattgtaataatcattgttattattattatacatattttgcttttaaatggataaggatttttaaggtatatgtaaactgtaaaacataaaatgcaaaatgccgtaagagacagtagtaataataatgattattatattgttatcattatctagcctgttttttcctgttttgtatttcttcctttaaatgctttcagaaatctgtatccccattcttcaccaccaccccacaacatttctgcttcttttcccatgccgggtcatgctaactttgaaagcttcagctctttccttcctcaatccttttcctggcacctctgatatgccttttgaaattcatgttaaagaatccctaggctgctatcacatgtggcatctttgttgagtacatgaataaatcaactggtgtgttttacgaaggatgattatgcttcattgtgggattgtatttttcttcttctatcacagggagaagtgaaatgacaacctcactagatacagttgagacctttggtaccacatcctactatgatgacgtgggcctgctctgtgaaaaagctgataccagagcactgatggcccagtttgtgcccccgctgtactccctggtgttcactgtgggcctcttgggcaatgtggtggtggtgatgatcctcataaaatacaggaggctccgaattatgaccaacatctacctgctcaacctggccatttcggacctgctcttcctcgtcacccttccattctggatccactatgtcagggggcataactgggtttttggccatggcatgtgtaagctcctctcagggttttatcacacaggcttgtacagcgagatctttttcataatcctgctgacaatcgacaggtacctggccattgtccatgctgtgtttgcccttcgagcccggactgtcacttttggtgtcatcaccagcatcgtcacctggggcctggcagtgctagcagctcttcctgaatttatcttctatgagactgaagagttgtttgaagagactctttgcagtgctctttacccagaggatacagtatatagctggaggcatttccacactctgagaatgaccatcttctgtctcgttctccctctgctcgttatggccatctgctacacaggaatcatcaaaacgctgctgaggtgccccagtaaaaaaaagtacaaggccatccggctcatttttgtcatcatggcggtgtttttcattttctggacaccctacaatgtggctatccttctctcttcctatcaatccatcttatttggaaatgactgtgagcggagcaagcatctggacctggtcatgctggtgacagaggtgatcgcctactcccactgctgcatgaacccggtgatctacgcctttgttggagagaggttccggaagtacctgcgccacttcttccacaggcacttgctcatgcacctgggcagatacatcccattccttcctagtgagaagctggaaagaaccagctctgtctctccatccacagcagagccggaactctctattgtgttttaggtcagatgcagaaaattgcctaaagaggaaggaccaaggagatgaagcaaacacattaagccttccacactcacctctaaaacagtccttcaaacttccagtgcaacactgaagctcttgaagacactgaaatatacacacagcagtagcagtagatgcatgtaccctaaggtcattaccacaggccaggggctgggcagcgtactcatcatcaaccctaaaaagcagagctttgcttctctctctaaaatgagttacctacattttaatgcacctgaatgttagatagttactatatgccgctacaaaaaggtaaaactttttatattttatacattaacttcagccagctattgatataaataaaacattttcacacaatacaataagttaactattttattttctaatgtgcctagttctttccctgcttaatgaaaagcttgttttttcagtgtgaataaataatcgtaagcaacataatggcatgccattcctgttctaaatattcttcatagtttgttgatttcctgttcacactgaatgacaaaatttcccatgggtgactcacagagccctgagaagtgtgcactgtcctcttaatgccaatctgacattgcagaggaggccagggcctcctgtacatatgaagtagcccgggtatggagcggggaggtgttgggcttgcagggactctcggcacctgtag

SEQ ID NO: 6 - protein, 355 aa, Homo sapiens
MTTSLDTVETFGTTSYYDDVGLLCEKADTRALMAQFVPPLYSLVFTVGLLGNVVVVMILIKYRRLRIMTNIYLLNLAISDLLFLVTLPFWIHYVRGHNWVFGHGMCKLLSGFYHTGLYSEIFFIILLTIDRYLAIVHAVFALRARTVTFGVITSIVTWGLAVLAALPEFIFYETEELFEETLCSALYPEDTVYSWRHFHTLRMTIFCLVLPLLVMAICYTGIIKTLLRCPSKKKYKAIRLIFVIMAVFFIFWTPYNVAILLSSYQSILFGNDCERSKHLDLVMLVTEVIAYSHCCMNPVIYAFVGERFRKYLRHFFHRHLLMHLGRYIPFLPSEKLERTSSVSPSTAEPELSIVF

SEQ ID NO: 7 - DNA, 1269 bp, Homo sapiens
  CDS (1)..(1083)
atgaaccccacggatatagcagataccaccctcgatgaaagcatatacagcaattactatctgtatgaaagtatccccaagccttgcaccaaagaaggcatcaaggcatttggggagctcttcctgcccccactgtattccttggtttttgtatttggtctgcttggaaattctgtggtggttctggtcctgttcaaatacaagcggctcaggtccatgactgatgtgtacctgctcaaccttgccatctcggatctgctcttcgtgttttccctccctttttggggctactatgcagcagaccagtgggtttttgggctaggtctgtgcaagatgatttcctggatgtacttggtgggcttttacagtggcatattctttgtcatggtcatgagcattgatagatacctggcaattgtgcacgcggtgttttccttgagggcaaggaccttgacttatggggtcatcaccagtttggctacatggtcagtggctgtgttcgcctcccttcctggctttctgttcagcacttgttatactgagcgcaaccatacctactgcaaaaccaagtactctctcaactccacgacgtggaaggttctcagctccctggaaatcaacattctcggattggtgatccccttagggatcatgctgttttgctactccatgatcatcaggaccttgcagcattgtaaaaatgagaagaagaacaaggcggtgaagatgatctttgccgtggtggtcctcttccttgggttctggacaccttacaacatagtgctcttcctagagaccctggtggagctagaagtccttcaggactgcacctttgaaagatacttggactatgccatccaggccacagaaactctggcttttgttcactgctgccttaatcccatcatctacttttttctgggggagaaatttcgcaagtacatcctacagctcttcaaaacctgcaggggcctttttgtgctctgccaatactgtgggctcctccaaatttactctgctgacacccccagctcatcttacacgcagtccaccatggatcatgatctccatgatgctctgtaggaaaaatgaaatggtgaaatgcagagtcaatgaacttttccacattcagagcttactttaaaattggtatttttaggtaagagattcctgagccagtgtcaggaggaaggcttacacccacagtggaaagacagcttctcatcctgcaggcagctttttctctcccactagacaagtccagcctgg

SEQ ID NO: 8 - protein, 360 aa, Homo sapiens [sequence omitted]

SEQ ID NO: 9 - DNA, 3383 bp, Homo sapiens
  CDS (55)..(1113)
agaagagctgagacatccgttcccctacaagaaactctccccgggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactggggtggatttggtttggaagtgagggtcagagaggagtcagagagaatccctagtcttcaagcagattggagaaacccttgaaaagacatcaagcacagaaggaggaggaggaggtttaggtcaagaagaagatggattggtgtaaaaggatgggtctggtttgcagagcttgaacacagtctcacccagactccaggctgtctttcactgaatgcttctgacttcatagatttccttcccatcccagctgaaatactgaggggtctccaggaggagactagatttatgaatacacgaggtatgaggtctaggaacatacttcagctcacacatgagatctaggtgaggattgattacctagtagtcatttcatgggttgttgggaggattctatgaggcaaccacaggcagcatttagcacatactacacattcaataagcatcaaactcttagttactcattcagggatagcactgagcaaagcattgagcaaaggggtcccatataggtgagggaagcctgaaaaactaagatgctgcctgcccagtgcacacaagtgtaggtatcattttctgcatttaaccgtcaataggcaaaggggggaagggacatattcatttggaaataagctgccttgagccttaaaacccacaaaagtacaatttaccagcctccgtatttcagactgaatgggggtggggggggcgccttaggtacttattccagatgccttctccagacaaaccagaagcaacagaaaaaatcgtctctccctccctttgaaatgaatataccccttagtgtttgggtatattcatttcaaagggagagagagaggtttttttctgttctttctcatatgattgtgcacatacttgagactgttttgaatttgggggatggctaaaaccatcatagtacaggtaaggtgagggaatagtaagtggtgagaactactcagggaatgaaggtgtcagaataataagaggtgctactgactttctcagcctctgaatatgaacggtgagcattgtggctgtcagcaggaagcaacgaagggaaatgtctttccttttgctcttaagttgtggagagtgcaacagtagcataggaccctaccctctgggccaagtcaaagacattctgacatcttagtatttgcatattcttatgtatgtgaaagttacaaattgcttgaaagaaaatatgcatctaataaaaaacaccttctaaaataaaaaaaaaaaaaaaaaaaaaaaaaaa

SEQ ID NO: 10 - protein, 352 aa, Homo sapiens [sequence omitted]

SEQ ID NO: 11 - DNA, 4018 bp, Homo sapiens
  CDS (553)..(1677)
tgcaaacgttcccaaatcttcccagtcggcttgcagagactccttgctcccaggagataaccaggtaaaggagtatgaaagtttgggtacaaactcattgctgcaaattgaaaaccatgcaaaggctgtcttcctctggggagttcaatgcctctctttttcttatcactttaccattggttggactttgattccagggatcctacgattactcaataccctacaggatatacatggttaaccatttgcatttgggcaaataggcgttacttttcaataggaagtggcaatccagaacttgcttttgggcaattctagtagctcaccgcttttttcttaatgactgctagaagctgcatcttattgacagatggtcatcacattggtgagctggagtcatcagattgtggggcccggagtgaggctgaagggagtggatcagagcactgcctgagagtcacctctactttcctgctaccgctgcctgtgagctgaaggggctgaaccatacactcctttttctacaaccagcttgcattttttctgcccacaatgagcggggaatcaatgaatttcagcgatgttttcgactccagtgaagattattttgtgtcagtcaatacttcatattactcagttgattctgagatgttactgtgctccttgcaggaggtcaggcagttctccaggctatttgtaccgattgcctactccttgatctgtgtctttggcctcctggggaatattctggtggtgatcacctttgctttttataagaaggccaggtctatgacagacgtctatctcttgaacatggccattgcagacatcctctttgttcttactctcccattctgggcagtgagtcatgccaccggtgcgtgggttttcagcaatgccacgtgcaagttgctaaaaggcatctatgccatcaactttaactgcgggatgctgctcctgacttgcattagcatggaccggtacatcgccattgtacaggcgactaagtcattccggctccgatccagaacactaccgcgcagcaaaatcatctgccttgttgtgtgggggctgtcagtcatcatctccagctcaacttttgtcttcaaccaaaaatacaacacccaaggcagcgatgtctgtgaacccaagtaccagactgtctcggagcccatcaggtggaagctgctgatgttggggcttgagctactctttggtttctttatccctttgatgttcatgatattttgttacacgttcattgtcaaaaccttggtgcaagctcagaattctaaaaggcacaaagccatccgtgtaatcatagctgtggtgcttgtgtttctggcttgtcagattcctcataacatggtcctgcttgtgacggctgcaaatttgggtaaaatgaaccgatcctgccagagcgaaaagctaattggctatacgaaaactgtcacagaagtcctggctttcctgcactgctgcctgaaccctgtgctctacgcttttattgggcagaagttcagaaactactttctgaagatcttgaaggacctgtggtgtgtgagaaggaagtacaagtcctcaggcttctcctgtgccgggaggtactcagaaaacatttctcggcagaccagtgagaccgcagataacgacaatgcgtcgtccttcactatgtgatagaaagctgagtctccctaaggcatgtgtgaaacatactcatagatgttatgcaaaaaaaagtctatggccaggtatgcatggaaaatgtgggaattaagcaaaatcaagcaagcctctctcctgcgggacttaacgtgctcatgggctgtgtgatctcttcagggtggggtggtctctgataggtagcattttccagcactttgcaaggaatgttttgtagctctagggtatatatccgcctggcatttcacaaaacagcctttgggaaatgctgaattaaagtgaattgttgacaaatgtaaacattttcagaaatattcatgaagcggtcacagatcacagtgtcttttggttacagcacaaaatgatggcagtggtttgaaaaactaaaacagaaaaaaaaatggaagccaacacatcactcattttaggcaaatgtttaaacatttttatctatcagaatgtttattgttgctggttataagcagcaggattggccggctagtgtttcctctcatttccctttgatacagtcaacaagcctgaccctgtaaaatggaggtggaaagacaagctcaagtgttcacaacctggaagtgcttcgggaagaaggggacaatggcagaacaggtgttggtgacaattgtcaccaattggataaagcagctcaggttgtagtgggccattaggaaactgtcggtttgctttgatttccctgggagctgttctctgtcgtgagtgtctcttgtctaaacgtccattaagctgagagtgctatgaagacaggatctagaataatcttgctcacagctgtgctctgagtgcctagcggagttccagcaaacaaaatggactcaagagagatttgattaatgaatcgtaatgaagttggggtttattgtacagtttaaaatgttagatgtttttaattttttaaataaatggaatactttttttttttttttaaagaaagcaactttactgagacaatgtagaaagaagttttgttccgtttctttaatgtggttgaagagcaatgtgtggctgaagacttttgttatgaggagctgcagattagctaggggacagctggaattatgctggcttctgataattattttaaaggggtctgaaatttgtgatggaatcagattttaacagctctcttcaatgacatagaaagttcatggaactcatgtttttaaagggctatgtaaatatatgaacattagaaaaatagcaacttgtgttacaaaaatacaaacacatgttaggaaggtactgtcatgggctaggcatggtggctcacacctgtaatcccagcattttgggaagctaagatgggtggatcacttgaggtcaggagtttgagaccagcctggccaacatggcgaaacccctctctactaaaaatacaaaaatttgccaggcgtggtggcgggtgcctgtaatcccagctacttgggaggctgaggcaagagaatcgcttgaacccaggaggcagaggttgcagtgagccgagatcgtgccattgcactccagcctgggtgacaaagcgagactccatctcaaaaaaaaaaaaaaaaaaaaaggaaagaactgtcatgtaaacataccaacatgtttaaacctgacaatggtgttatttgaaactttatattgttcttgtaagctttaactatatctctctttaaaatgcaaaataatgtcttaagattcaaagtctgtatttttaaagcatggctttggctttgcaaaataaaaaatgtgttttgtacatgaagtaggaatcgtatttcagcttcaaggttcagattgaggggcccactgtttggagaggatggtattcaggctttctcatgtccttcaaatctgttagcgtttgactctagaaatcaaagcaaaggagtggttacccagacacttcttttggtgtgatcaatgcgctgatgtgatctatgaagatgattcatgcttgaaaactagcacagaaacatcttgcttatttgccaaagctgggagatgagcttctctgcataatttaaatgttcagataaatgaagctgacttatttaagcaataaccttttaaacattttagctaagatgtataaaaatgtttccaaaatataccacatactttatttcttcttaaatgtagtacattaggttacatcatttttcttgctgtcttgggcatcaaaacaggtgccatggtaacctgacactctcaggagacattaagatagaaggggctgttcttcagtggttcccattgattctccccatatctttttgctctcaggctctggccgtctcttcctgagccttaactgtgt

SEQ ID NO: 12 - protein, 374 aa, Homo sapiens
MSGESMNFSDVFDSSEDYFVSVNTSYYSVDSEMLLCSLQEVRQFSRLFVPIAYSLICVFGLLGNILVVITFAFYKKARSMTDVYLLNMAIADILFVLTLPFWAVSHATGAWVFSNATCKLLKGIYAINFNCGMLLLTCISMDRYIAIVQATKSFRLRSRTLPRSKIICLVVWGLSVIISSSTFVFNQKYNTQGSDVCEPKYQTVSEPIRWKLLMLGLELLFGFFIPLMFMIFCYTFIVKTLVQAQNSKRHKAIRVIIAVVLVFLACQIPHNMVLLVTAANLGKMNRSCQSEKLIGYTKTVTEVLAFLHCCLNPVLYAFIGQKFRNYFLKILKDLWCVRRKYKSSGFSCAGRYSENISRQTSETADNDNASSFTM

SEQ ID NO: 13 - DNA, 2139 bp, Homo sapiens
  CDS (64)..(1200)
agacaggggtagtgcgaggccgggcacagccttcctgtgtggttttaccgcccagagagcgtcatggacctggggaaaccaatgaaaagcgtgctggtggtggctctccttgtcattttccaggtatgcctgtgtcaagatgaggtcacggacgattacatcggagacaacaccacagtggactacactttgttcgagtctttgtgctccaagaaggacgtgcggaactttaaagcctggttcctccctatcatgtactccatcatttgtttcgtgggcctactgggcaatgggctggtcgtgttgacctatatctatttcaagaggctcaagaccatgaccgatacctacctgctcaacctggcggtggcagacatcctcttcctcctgacccttcccttctgggcctacagcgcggccaagtcctgggtcttcggtgtccacttttgcaagctcatctttgccatctacaagatgagcttcttcagtggcatgctcctacttctttgcatcagcattgaccgctacgtggccatcgtccaggctgtctcagctcaccgccaccgtgcccgcgtccttctcatcagcaagctgtcctgtgtgggcatctggatactagccacagtgctctccatcccagagctcctgtacagtgacctccagaggagcagcagtgagcaagcgatgcgatgctctctcatcacagagcatgtggaggcctttatcaccatccaggtggcccagatggtgatcggctttctggtccccctgctggccatgagcttctgttaccttgtcatcatccgcaccctgctccaggcacgcaactttgagcgcaacaaggccatcaaggtgatcatcgctgtggtcgtggtcttcatagtcttccagctgccctacaatggggtggtcctggcccagacggtggccaacttcaacatcaccagtagcacctgtgagctcagtaagcaactcaacatcgcctacgacgtcacctacagcctggcctgcgtccgctgctgcgtcaaccctttcttgtacgccttcatcggcgtcaagttccgcaacgatctcttcaagctcttcaaggacctgggctgcctcagccaggagcagctccggcagtggtcttcctgtcggcacatccggcgctcctccatgagtgtggaggccgagaccaccaccaccttctccccataggcgactcttctgcctggactagagggacctctcccagggtccctggggtggggatagggagcagatgcaatgactcaggacatccccccgccaaaagctgctcagggaaaagcagctctcccctcagagtgcaagcccctgctccagaagatagcttcaccccaatcccagctacctcaaccaatgccaaaaaaagacagggctgataagctaacaccagacagacaacactgggaaacagaggctattgtcccctaaaccaaaaactgaaagtgaaagtccagaaactgttcccacctgctggagtgaaggggccaaggagggtgagtgcaaggggcgtgggagtggcctgaagagtcctctgaatgaaccttctggcctcccacagactcaaatgctcagaccagctcttccgaaaaccaggccttatctccaagaccagagatagtggggagacttcttggcttggtgaggaaaagcggacatcagctggtcaaacaaactctctgaacccctccctccatcgttttcttcactgtcctccaagccagcgggaatggcagctgccacgccgccctaaaagcacactcatcccctcacttgccgcgtcgccctcccaggctctcaacaggggagagtgtggtgtttcctgcaggccaggccagctgcctccgcgtgatcaaagccacactctgggctccagagtggggatgacatgcactcagctcttggctccactgggatgggaggagaggacaagggaaatgtcaggggcggggagggtgacagtggccgcccaaggcccacgagcttgttctttgttctttgtcacagggactgaaaacctctcctcatgttctgctttcgattcgttaagagagcaacattttacccacacacagataaagttttcccttgaggaaacaacagctttaaaag

SEQ ID NO: 14 - protein, 378 aa, Homo sapiens
MDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 15 - DNA, 2372 bp, Homo sapiens
  CDS (121)..(1188)
ctccagagaggctgctgctcattgagctgcactcacatgaggatacagactttgtgaagaaggaattggcaacactgaaacctccagaacaaaggctgtcactaaggtcccgctgccttgatggattatacacttgacctcagtgtgacaacagtgaccgactactactaccctgatatcttctcaagcccctgtgatgcggaacttattcagacaaatggcaagttgctccttgctgtcttttattgcctcctgtttgtattcagtcttctgggaaacagcctggtcatcctggtccttgtggtctgcaagaagctgaggagcatcacagatgtatacctcttgaacctggccctgtctgacctgctttttgtcttctccttcccctttcagacctactatctgctggaccagtgggtgtttgggactgtaatgtgcaaagtggtgtctggcttttattacattggcttctacagcagcatgtttttcatcaccctcatgagtgtggacaggtacctggctgttgtccatgccgtgtatgccctaaaggtgaggacgatcaggatgggcacaacgctgtgcctggcagtatggctaaccgccattatggctaccatcccattgctagtgttttaccaagtggcctctgaagatggtgttctacagtgttattcattttacaatcaacagactttgaagtggaagatcttcaccaacttcaaaatgaacattttaggcttgttgatcccattcaccatctttatgttctgctacattaaaatcctgcaccagctgaagaggtgtcaaaaccacaacaagaccaaggccatcaggttggtgctcattgtggtcattgcatctttacttttctgggtcccattcaacgtggttcttttcctcacttccttgcacagtatgcacatcttggatggatgtagcataagccaacagctgacttatgccacccatgtcacagaaatcatttcctttactcactgctgtgtgaaccctgttatctatgcttttgttggggagaagttcaagaaacacctctcagaaatatttcagaaaagttgcagccaaatcttcaactacctaggaagacaaatgcctagggagagctgtgaaaagtcatcatcctgccagcagcactcctcccgttcctccagcgtagactacattttgtgaggatcaatgaagactaaatataaaaaacattttcttgaatggcatgctagtagcagtgagcaaaggtgtgggtgtgaaaggtttccaaaaaaagttcagcatgaaggatgccatatatgttgttgccaacacttggaacacaatgactaaagacatagttgtgcatgcctggcacaacatcaagcctgtgattgtgtttattgatgatgttgaacaagtggtaactttaaaggattctgtatgccaagtgaaaaaaaaagatgtctgacctccttacatatgcaaaaatataccttcagagactgtcagtaggctggaagaagtggatattgaagttttgacatcaatgatgaggctccagttgtctattcattgactgatggtgaaatggctggagtgattctgaatcaaggtgattgtgattatagtgacaatgaagatgatgctattaatactgcataaaaagtgcctgtagatgacatggtgaaaatatttgacaggcttatggaaggactacagcagcatgcattcataacagaacaagaaattatgtcagcttataaaatcaaacagagacttctagacaaaaaccattgttgatgaggcagatgcctctagaagagacgtttaaaagccatcaaacacaatgcctcatcttccctggaggacccacttcctgatccctcaactgtgtctgatgtttcttctcatgtaagaaataaaaaataaaaataaaataaaatatattggtatgtaactacaggaaaaaaataaaaaatatatagtggacagtaacctttcaatcaaaactcagtatcataagtagagactgaaaacttgccgttattgattgttgttattaacagctgatacaggtattctgctgatgctactgctgcctagttaccatgaacacgttttttcactattaatggtgcgtcatattttttacttttaagtacttacgtgtgagtaagtgtaagaaaatgattgcttatcagtagtatcaatgatttactcaatatctgaatcaccttgattcagaaccatttcagctgtttcaccatcagtcaatgaataacagcctcattgatgtcaaaaacttcaatatccacttctttcagcctactgtagactctggaagtatactttttgcatatgtaaggaagtcagacttttttttt

SEQ ID NO: 16 - protein, 355 aa, Homo sapiens
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 17 - DNA, 3101 bp, Homo sapiens
  CDS (88)..(1155)
actcgtctctggtaaagtctgagcaggacagggtggctgactggcagatccagaggttcccttggcagtccacgccaggccttcaccatggatcagttccctgaatcagtgacagaaaactttgagtacgatgatttggctgaggcctgttatattggggacatcgtggtctttgggactgtgttcctgtccatattctactccgtcatctttgccattggcctggtgggaaatttgttggtagtgtttgccctcaccaacagcaagaagcccaagagtgtcaccgacatttacctcctgaacctggccttgtctgatctgctgtttgtagccactttgcccttctggactcactatttgataaatgaaaagggcctccacaatgccatgtgcaaattcactaccgccttcttcttcatcggcttttttggaagcatattcttcatcaccgtcatcagcattgataggtacctggccatcgtcctggccgccaactccatgaacaaccggaccgtgcagcatggcgtcaccatcagcctaggcgtctgggcagcagccattttggtggcagcaccccagttcatgttcacaaagcagaaagaaaatgaatgccttggtgactaccccgaggtcctccaggaaatctggcccgtgctccgcaatgtggaaacaaattttcttggcttcctactccccctgctcattatgagttattgctacttcagaatcatccagacgctgttttcctgcaagaaccacaagaaagccaaagccattaaactgatccttctggtggtcatcgtgtttttcctcttctggacaccctacaacgttatgattttcctggagacgcttaagctctatgacttctttcccagttgtgacatgaggaaggatctgaggctggccctcagtgtgactgagacggttgcatttagccattgttgcctgaatcctctcatctatgcatttgctggggagaagttcagaagatacctttaccacctgtatgggaaatgcctggctgtcctgtgtgggcgctcagtccacgttgatttctcctcatctgaatcacaaaggagcaggcatggaagtgttctgagcagcaattttacttaccacacgagtgatggagatgcattgctccttctctgaagggaatcccaaagccttgtgtctacagagaacctggagttcctgaacctgatgctgactagtgaggaaagatttttgttgttatttcttacaggcacaaaatgatggacccaatgcacacaaaacaaccctagagtgttgttgagaattgtgctcaaaatttgaagaatgaacaaattgaactctttgaatgacaaagagtagacatttctcttactgcaaatgtcatcagaactttttggtttgcagatgacaaaaattcaactcagactagtttagttaaatgagggtggtgaatattgttcatattgtggcacaagcaaaagggtgtctgagccctcaaagtgaggggaaaccagggcctgagccaagctagaattccctctctctgactctcaaatcttttagtcattatagatcccccagactttacatgacacagctttatcaccagagagggactgacacccatgtttctctggccccaagggcaaaattcccagggaagtgctctgataggccaagtttgtatcaggtgcccatccctggaaggtgctgttatccatggggaagggatatataagatggaagcttccagtccaatctcatggagaagcagaaatacatatttccaagaagttggatgggtgggtactattctgattacacaaaacaaatgccacacatcacccttaccatgtgcctgatccagcctctcccctgattacaccagcctcgtcttcattaagccctcttccatcatgtccccaaacctgcaagggctccccactgcctactgcatcgagtcaaaactcaaatgcttggcttctcatacgtccaccatggggtcctaccaatagattccccattgcctcctccttcccaaaggactccacccatcctatcagcctgtctcttccatatgacctcatgcatctccacctgctcccaggccagtaagggaaatagaaaaaccctgcccccaaataagaagggatggattccaaccccaactccagtagcttgggacaaatcaagcttcagtttcctggtctgtagaagagggataaggtacctttcacatagagatcatcctttccagcatgaggaactagccaccaactcttgcaggtctcaacccttttgtctgcctcttagacttctgctttccacacctggcactgctgtgctgtgcccaagttgtggtgctgacaaagcttggaagagcctgcaggtgctgctgcgtggcatagcccagacacagaagaggctggttcttacgatggcacccagtgagcactcccaagtctacagagtgatagccttccgtaacccaactctcctggactgccttgaatatcccctcccagtcaccttgtggcaagcccctgcccatctgggaaaataccccatcattcatgctactgccaacctggggagccagggctatgggagcagcttttttttcccccctagaaacgtttggaacaatctaaaagtttaaagctcgaaaacaattgtaataatgctaaagaaaaagtcatccaatctaaccacatcaatattgtcattcctgtattcacccgtccagaccttgttcacactctcacatgtttagagttgcaatcgtaatgtacagatggttttataatctgatttgttttcctcttaacgttagaccacaaatagtgctcgctttctatgtagtttggtaattatcattttagaagactctaccagactgtgtattcattgaagtcagatgtggtaactgttaaattgctgtgtatctgatagctctttggcagtctatatgtttgtataatgaatgagagaataagtcatgttccttcaagatcatgtaccccaatttacttgccattactcaattgataaacatttaacttgtttccaatgtttagcaaatacatattttatagaacttc

SEQ ID NO: 18 - protein, 355 aa, Homo sapiens
MDQFPESVTENFEYDDLAEACYIGDIVVFGTVFLSIFYSVIFAIGLVGNLLVVFALTNSKKPKSVTDIYLLNLALSDLLFVATLPFWTHYLINEKGLHNAMCKFTTAFFFIGFFGSIFFITVISIDRYLAIVLAANSMNNRTVQHGVTISLGVWAAAILVAAPQFMFTKQKENECLGDYPEVLQEIWPVLRNVETNFLGFLLPLLIMSYCYFRIIQTLFSCKNHKKAKAIKLILLVVIVFFLFWTPYNVMIFLETLKLYDFFPSCDMRKDLRLALSVTETVAFSHCCLNPLIYAFAGEKFRRYLYHLYGKCLAVLCGRSVHVDFSSSESQRSRHGSVLSSNFTYHTSDGDALLLL

SEQ ID NO: 19 - DNA, 1679 bp, Homo sapiens
  CDS (89)..(1147)
gtttgttggctgcggcagcaggtagcaaagtgacgccgagggcctgagtgctccagtagccaccgcatctggagaaccagcggttaccatggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttacttagtgttatg

SEQ ID NO: 20 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS